(19) United States
(12) Patent Application Publication
(10) Pub. No.: US 2004/0248104 A1
(43) Pub. Date: Dec. 9, 2004

(54) Methods and reagents for profiling quantities of nucleic acids

(76) Inventors: Zohar Yakhini, Ramat HaSharon (IL); Jeffrey R. Sampson, San Francisco, CA (US); Mel N. Kronick, Palo Alto, CA (US); Joel Myerson, Berkeley, CA (US); Anya Tsalenko, Chicago, IL (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/455,198

(22) Filed: Jun. 5, 2003

(51) Int. Cl. C12Q 1/68; C12P 19/34

(52) U.S. Cl. 435/6; 435/91.2

(57) ABSTRACT

Methods and reagents are disclosed for quantitatively analyzing a set of target nucleic acid sequences. In the method a unique set of oligonucleotide probe precursors is hybridized to the target nucleic acid sequences to produce hybrids. The hybrids are processed to alter the mass of each of the oligonucleotide probe precursors in the hybrids in a target sequence-mediated reaction to produce oligonucleotide products, each of which has a unique mass that is not a result of the presence of a mass tag in the oligonucleotide product. The processing of the hybrids may involve polymerase extension or ligation. The products are analyzed by means of mass spectrometry and the results are related to the amount of the target nucleic acid sequences in the set. Kits for carrying out the above methods are also disclosed.

### FIELD OF THE INVENTION

This invention relates to methods and reagents for conducting quantitation of nucleic acids such as, for example, gene expression profiling, by means of mass spectrometry.

### BACKGROUND OF THE INVENTION

Determining the nucleotide sequence and the expression levels of nucleic acids (DNA and RNA) is critical to understanding the function and control of genes and their relationship, for example, to disease discovery and disease management. Analysis of genetic information plays a crucial role in the biological experimentation. This has become especially true with regard to studies directed at understanding the fundamental genetic and environmental factors associated with disease and the effects of potential therapeutic agents on the cell. This paradigm shift has lead to an increasing need within the life science industries for more sensitive, more accurate and higher-throughput technologies for performing analysis on genetic material obtained from a variety of biological sources.

In any living cell that undergoes a biological process, different subsets of the total set of genes encoded in the organism's genome are expressed in different stages of the process. The particular subset expressed at a given stage and its quantitative composition is of extreme importance. Being able to measure subsets of genes that express themselves in different stages, different cells, and different organisms is instrumental in understanding biological processes. Such information can help the characterization of sequence to function relationships, the determination of effects (and side effects) of experimental treatments, and the understanding of many other molecular biological processes. Many disease states are characterized by differences in the expression levels of various genes either through changes in the copy number of genetic DNA or through changes in levels of transcription of particular genes. For example, losses and gains of genetic material play an important role in malignant transformation and progression. Changes in the expression (transcription) levels of particular genes such as oncogenes or tumor suppressors serve as signposts for the presence and progression of various cancers. Control of the cell cycle and cell development as well as diseases is characterized by the variations in the transcription levels of particular genes. Thus, for example, a viral infection is often characterized by the elevated expression of genes of the particular virus and of the genes that the host activates in reaction to the infection.

The purpose of a gene expression profiling assay is to measure the expression levels of a set of genes in a mixture. Given the huge amount of sequence information accumulated by the human genome project, the rate at which such information is still being generated, and the extent of gene hunting efforts currently invested by the scientific community, it is reasonable to approach gene expression profiling assuming complete knowledge of the sequences of the genes of interest.

Several approaches to gene expression profiling are known in the art. One such approach is a Northern blot wherein mRNA's from a cellular sample are separated on an electrophoretic gel and then blotted onto a membrane. The membrane is then probed with a specific DNA or RNA probe to ascertain the molecular weight and the quantity of mRNA's complementary to the probe. The biochemical manipulations are significant and usually only one gene or a small set of genes can be studied with each probing and detection. Thus, Northern blotting is not well suited to the study of large numbers of genes.

Another approach involves cDNA arrays. Arrays of cDNA's or arrays of PCR products from a set of specific cDNA's are applied to membranes or non-porous surfaces in a well-defined two-dimensional format. The arrays are then hybridized to the mixture of mRNA's present in a sample that have been labeled with a radioactive or fluorescent tag. The strength of the signal at each location on the array indicates the amount of the mRNA that corresponds to the gene from which the cDNA comes. By labeling two different mRNA populations with different fluorescent tags, differential gene expression measurements can be made. Specific arrays do need to be created for the particular set of genes that is being studied. This can be tedious and expensive depending on the number of genes because each feature on the array corresponds to a particular, well-characterized clone. Once formed, the arrays need to be hybridized to target, washed, and scanned so that significant sample work-up is required.

Another approach concerns oligonucleotide arrays. These arrays are similar to cDNA arrays except that the arrays consist of oligonucleotides (usually less than 35 nucleotides in length) that are either synthesized in situ or, alternatively, deposited in the array format. Specific arrays do need to be created for the particular set of genes that is being studied. This can be tedious and expensive depending on the method of array creation and the number of genes. Once formed the arrays need to be hybridized to target, washed, and scanned so that significant sample work-up is required.

Another approach is known as differential display; mRNA molecules are converted biochemically into a series of unique length pieces of DNA (usually through use of restriction enzymes and/or PCR), are labeled, and then are separated on high resolution electrophoretic gels. The biochemical process is designed so that each specific mRNA sequence turns into a fragment with a unique length and/or color tag. Typically, samples to be compared are run in neighboring lanes of the gel. Since there is nominally a 1:1 relationship between bands on the gel and mRNA from specific genes, comparisons of band intensities from one sample to another can yield information about genes that have increased or decreased in level of expression. This has been valuable as a screening technique but the identification of particular bands with a particular gene is usually very tedious and involves careful extraction of a narrow band from the gel followed by sequencing or probing. Once identified, a particular band can be linked to a particular gene in other studies but unless it is sequenced or probed, the linkage is merely inferred.

Another approach is designated as SAGE (Serial Analysis of Gene Expression). This technique relies on the use of a clever cloning technique to concatenate short sequence tags, each of which (almost) uniquely defines a particular mRNA molecule from which the tag is derived. The concatenated tags are then sequenced using conventional dideoxy sequencing technology. The population of tags present from a particular sample is counted and thus gives the distribution of mRNA molecules (and thus genes), which are expressed in the sample. The technique is very powerful for screening for changes as a first pass screen of an entire mRNA population but because of the enormous sample preparation steps, it does not appear very desirable for detailed studies of particular sets of genes.

Another approach is known as MPSS (Massively Parallel Simple Sequencing). This technique also relies on the use of a clever cloning technique using short sequence tags each of which (almost) uniquely defines a particular mRNA molecule from which the tag is derived. In MPSS, each tag becomes associated with a particular bead that is sequenced using a novel degradation technique that can occur in a massively parallel fashion. The sequence determined to be present on each bead determines the gene from which the mRNA is transcribed. The number of beads with a particular sequence is a direct measure of its abundance in the mRNA pool from the original sample. The technique is very powerful for screening for changes as a first pass but because of the sample preparation steps, it does not appear very desirable for detailed studies of particular sets of genes.

Another approach is designated as RT-PCR. Gene specific oligonucleotide primers are utilized to amplify a specific PCR fragment from a particular gene. The quantity of the PCR fragment is measured either by the intensity of a gel electrophoresis band or by use of an in-situ PCR measurement such as TaqMan. This technique is very sensitive and has a wide dynamic range and requires very little user manipulation. Each gene to be analyzed requires separate primers and separate PCR's that makes this technique very expensive and cumbersome for the analysis of large numbers of genes.

Another approach is known as an RNAse Protection Assay. Antisense RNA probes to specific RNA sequences are created. The lengths of the probes are defined so that a particular length corresponds to a particular sequence. A finite set of probes is hybridized in solution to the target mixture containing the RNA's of interest. Probe-target heteroduplexes form when there is target for the probe. RNAse is added and it chews up all single stranded probe and target. The remaining double stranded probe-target heteroduplexes are separated by gel electrophoresis and then detected after blotting using a label on the probe for detection. The method is great for small numbers of targets since each target corresponds to a particular length of double-stranded RNA but the technique does involve lots of steps including gel electrophoresis and blotting (unless the detection is in situ such as by fluorescence.)

The methods discussed above for gene expression profiling assays suffer from several shortcomings. Hybridization array based assays are time consuming (i.e., using the current technology, the hybridization step can take up to 16 hours with the subsequent wash and scanning steps taking an additional 2 hours). Specific arrays have to be designed and manufactured for every given set of genes. Manufacturing of these arrays is significantly more complicated than the synthesis, in solution, of DNA probes. SAGE suffers from great inaccuracies. PCR based assays are very labor intensive whenever more than a few genes need to be interrogated.

Mass spectrometry (MS) is a powerful tool for analyzing complex mixtures of compounds, including nucleic acids. In addition to accurately determining an intact mass, primary structure information can be obtained by several different MS strategies. The use of MS for DNA analysis has potential application to the detection of DNA modifications, DNA fragment mass determination, and DNA sequencing (see for example; Fields, G.B., Clinical Chemistry 43, 1108 (1997)). Both fast atom bombardment (FAB) and electrospray ionization (ESI) collision-induced dissociation/tandem MS have been applied for identification of DNA modification sites.

Although MS is a powerful tool for analyzing complex mixtures of related compounds, including nucleic acids, its utility for analyzing the sequence of nucleic acids is limited by available ionization and detection methods. For example, ESI mass spectrometry produces a distribution of highly charged ions having a mass-to-charge ratio in the range of commercially available quadrupole mass analyzers. While ESI MS is sensitive, requiring only femtomole quantities of sample, it relies on multiple charges to achieve efficient ionization and produces complex and difficult-to-interpret multiply-charged spectra for even simple nucleic acids.

Matrix-assisted laser desorption ionization (MALDI) used in conjunction with a time-of-flight (TOF) mass analyzer holds great potential for sequencing nucleic acids because of its relatively broad mass range, high resolution (m/Dm<1.0 at mass 5,000) and sampling rate (up to 1 sample/second). In addition, TOF analyzers are suitable for large dynamic range measurements (10³-10⁴), which allows for quantitative analysis of analyte mixtures having a broad concentration range. In one aspect MALDI offers a potential advantage over ESI and FAB in that biomolecules of large mass can be ionized and analyzed readily. Furthermore, in contrast to ESI, MALDI produces predominantly singly charged species.

However, in general, MALDI analysis of DNA may suffer from lack of resolution of high molecular weight DNA fragments, DNA instability, and interference from sample preparation reagents. Longer oligonucleotides can give broader, less-intense signals, because MALDI imparts greater kinetic energies to ions of higher molecular weights. Although it may be used to analyze high molecular-weight nucleic acids, MALDI-TOF can induce cleavage of the nucleic acid backbone, which further complicates the resulting spectrum. As a result, the length of nucleic acid sequences that may be analyzed via MALDI-TOF has been limited to about 100 bases or residues. Recent progress in infrared MALDI-TOF appears to have overcome some of these limitations.

Efforts have been made to address some of the aforementioned deficiencies with mass spectroscopic analyses of nucleic acids. For example, Wang et al. (WO 98/03684) have taken advantage of “in source fragmentation” and coupled it with delayed pulsed ion extraction methods for analyzing nucleic acid analytes. Gut (WO 96/27681) on the other hand discloses methods for altering the charge properties of the phosphodiester backbone of nucleic acids in ways that make them more stable and hence more amenable to MS analyses. Methods for introducing modified nucleotides that stabilize the nucleic acid against fragmentation have also been described (Schneider and Chait, Nucleic Acids Res, 23, 1570 (1995), Tang et al., J Am Soc Mass Spectrom, 8, 218-224, 1997).

Koster (U.S. Pat. No. 5,547,835) has developed methodologies for multiplexing nucleic acid analysis in order to increase sample throughput. This approach involves introducing mass modifications into the interrogating oligonucleotide probe, nucleoside triphosphates, as well as using integrated mass-tag sequences that allow multiplexing by hybridization of tag specific probes with mass differentiated molecular weights.

Cleavable mass-tags have also been exploited to address some of the problems associated with MS analysis of nucleic acids. For example, PCT Application WO 95/04160 (Southern, et al.) discloses an indirect method for analyzing the sequence of target nucleic acids using target-mediated ligation between a surface-bound DNA probe and cleavable mass-tagged oligonucleotides containing reporter groups using mass spectrometric techniques. The sequence to be determined is first hybridized to an oligonucleotide attached to a solid support. The solid support carrying the hybrids from above is incubated with a solution of coded oligonucleotide reagents that form a library comprising all sequences of a given length. Ligase is introduced so that the oligonucleotide on the support is ligated to the member of the library that is hybridized to the target adjacent the oligonucleotide. Non-ligated reagents are removed by washing. A linker that is part of the member of the library ligated to the oligonucleotide is broken to detach a tag, which is recovered and analyzed by mass spectrometry.

One common focus of the above technologies is to provide methods for increasing the number of target sites (either intra- or inter-target) that can be interrogated in a single determination where some portion of the target sequence is known. That is, for a given set of targets a set of probes/precursors is sought for so that distinguishable features in the output mass-spectrum will correspond to different target sequences in the set. This requires mass differentiation that can be obtained by cleavable or non-cleavable mass tags or by internal mass modifications of the probe/precursor oligonucleotides. This multiplexing theme is either directly stated or implied in the teachings of the above patent applications. None of the teachings or the claims, however, describes an algorithmic or a heuristic approach to optimizing the multiplexing scheme and none indicate the extent to which such multiplexing can theoretically work. One component of the current invention consists of a procedure for designing such multiplexing schemes for gene expression profiling assays.

In Howbert, et al., WO 97/27327, methods are described for the use of mass-tagged probes for mass spectrometry based gene expression analysis. A unique chemical mass tag is developed for every gene in the set of genes of interest. Then, a probe (or possibly more than one) for this gene is designed, which should be specific (in terms of cross hybridization to the background message). Mass tags are attached to the probes by photo cleavable linkers. The target molecules are immobilized on a solid support. After hybridizing and washing, the tags are cleaved and the tag mixture is analyzed by mass spectrometry. Expression levels of the genes of interest are determined from the resulting spectrum. Immobilizing the target is complicated and potentially costly. It might also skew quantities. It is, however, a necessary step for the process of Howbert, et al., as the target bound probes need to be separated from the rest of the probe mixture. The current invention uses the mass to register the probe-target hybridization. It therefore allows for the entire reaction to take place in solution phase. Embodiments that in addition utilize arrays have a better information content but the array step is not crucial.

### SUMMARY OF THE INVENTION

One embodiment of the present invention is directed to a method of quantitatively analyzing a set of target nucleic acid sequences. A set of oligonucleotide probe precursors is hybridized to the target nucleic acid sequences to produce hybrids. A unique set of oligonucleotide precursors is employed for each set of target nucleic acid sequences. The hybrids are processed to alter the mass of each of the oligonucleotide Probe precursors in the hybrids in a target sequence-mediated reaction to produce oligonucleotide products. Each of the oligonucleotide products has a unique mass characteristic of its respective target nucleic acid sequence. The unique mass is not a result of the presence of a mass tag in the oligonucleotide product. The oligonucleotide products are analyzed by means of mass spectrometry. The results thereof are related to the amount of the target nucleic acid sequences in the set.

Another embodiment of the present invention is a method of determining the expression of genes in a set of genes. A set of oligonucleotide probe precursors is hybridized to the set of genes to produce hybrids. The composition of each of the genes is known to the extent necessary to select the set of specific oligonucleotide probe precursors. Each of the oligonucleotide probe precursors has a unique mass, binds specifically to a respective gene, is substantially incapable of hybridizing to another of the oligonucleotide probe precursors to produce a hybrid capable of enzymatic extension, and is modifiable by enzymatic extension to yield oligonucleotide products. Each of the oligonucleotide products has a unique mass that is not a result of the presence of a mass tag in the oligonucleotide product. Each of the hybridized oligonucleotide probe precursors is modified by polymerizing at least one nucleotide at the 3′-end of the hybridized oligonucleotide probe precursors to produce the oligonucleotide products, which are analyzed by means of mass spectrometry. The results thereof are related to the expression of the genes in the set.

Another embodiment of the present invention is a method of determining the expression of genes in a set of genes. A set of oligonucleotide probe precursors is hybridized to the genes to produce hybrids. The composition of each of the genes is known to the extent necessary to select the set of oligonucleotide probe precursors. This set is selected so that at least two, usually two, or at least three, usually three, of the oligonucleotide probe precursors in the set bind specifically and adjacently to a respective gene and are ligatable to yield oligonucleotide products. Each of the oligonucleotide products has a different mass that is not a result of the presence of a mass tag in the oligonucleotide product. Adjacent oligonucleotide probe precursors are ligated to produce oligonucleotide products, each of which has a unique mass characteristic of its respective gene. The oligonucleotide products are analyzed by means of mass spectrometry and the results thereof are related to the amount of expression of the genes in the set.

Another embodiment of the present invention is a composition comprising a set of oligonucleotide probe precursors. Each of the oligonucleotide probe precursors in the set binds specifically to a respective target nucleic acid sequence. The oligonucleotide probe precursors are substantially incapable of hybridizing to one another to produce hybrids capable of enzymatic extension. The oligonucleotide probe precursors can be mass modified by enzymatic extension to yield oligonucleotide products each having a different mass that is not a result of the presence of a mass tag in the oligonucleotide product.

Another embodiment of the present invention is a kit for analyzing a set of target nucleic acid sequences. The kit comprises in packaged combination a composition as described above, an enzyme having DNA polymerase activity and chain-terminating nucleotide triphosphates.

Another embodiment of the present invention is a composition comprising a set of oligonucleotide probe precursors. Each of the oligonucleotide probe precursors has a length of about 5 to about 10 nucleotides. At least three of the oligonucleotide probe precursors in the set bind specifically to a respective target nucleic acid sequence and are ligatable to yield an oligonucleotide product having a different mass that is not a result of the presence of a mass tag in the oligonucleotide product.

Another embodiment of the present invention is a kit for analyzing a set of target nucleic acid sequences. The kit comprises in packaged combination a composition as described above and a DNA ligase or a condensing agent for ligating the oligonucleotide probe precursors.

Another embodiment of the present invention is a method of determining the expression of genes in a set of genes. A set of genes is hybridized to a multiplicity of nucleic acid probes attached to a surface in an array. The multiplicity of nucleic acid probes comprise a cleavable linker- attached to the surface and a nucleic acid sequence having a 3′-end and a terminal 5′-phosphate wherein the 3′-end of the nucleic acid sequence is attached to the cleavable linker. A set of oligonucleotide probe precursors to the genes. A unique set of oligonucleotide precursors is employed for each set of genes. The hybrids are processed to alter the mass of each of the oligonucleotide probe precursors in the hybrids in a target sequence-mediated reaction to produce oligonucleotide products. Each of the oligonucleotide products has a unique mass that is not a result of the presence of a mass tag in the oligonucleotide product. The cleavable linker is cleaved and the oligonucleotide products are analyzed by mass spectrometry. The results are related to the amount of the genes in the set.

### BRIEF DESCRIPTION OF THE DRAWINGS

FIG. 1 is a schematic drawing depicting the components of an ETML in accordance with the present invention.

FIG. 2 is a schematic drawing depicting the reaction of the components of the ETML of FIG. 1.

FIG. 3 is a schematic drawing depicting the product of the reaction of the components of the ETML of FIG. 1.

FIG. 4 is a schematic drawing depicting the mass spectrum of the products of the components of the ETML of FIG. 1.

FIG. 5 is a schematic drawing depicting and example of probe to probe priming in ETME.

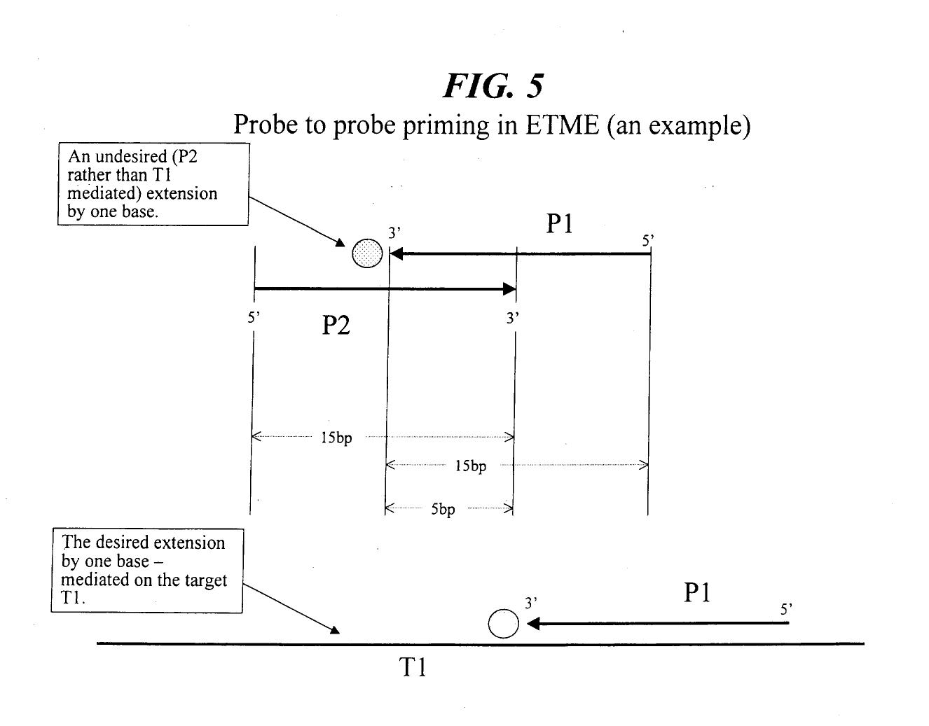

FIG. 6 is a schematic drawing depicting a pair of oligonucleotides comprising a hybridized portion.

### DETAILED DESCRIPTION OF THE INVENTION

Definitions

The term “polynucleotide” or “nucleic acid” refers to a compound or composition that is a polymeric nucleotide or nucleic acid polymer. The polynucleotide may be a natural compound or a synthetic compound. The polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. It is thus obvious that isolation of a polynucleotide from the natural state often results in fragmentation. It may be useful to fragment longer target nucleic acid sequences, particularly RNA, prior to hybridization to reduce competing intramolecular structures.

The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, CDNA, and the like.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method. For purposes of this invention, the polynucleotide, or a cleaved fragment obtained from the polynucleotide, will usually be at least partially denatured or single stranded or treated to render it denatured or single stranded. Such treatments are well known in the art and include, for instance, heat or alkali treatment, or enzymatic digestion of one strand. For example, dsDNA can be heated at 90 to 100° C. for a period of about 1 to 10 minutes to produce denatured material.

The nucleic acids may be generated by in vitro replication and/or amplification methods such as the Polymerase Chain Reaction (PCR), asymmetric PCR, the Ligase Chain Reaction (LCR) and so forth. The nucleic acids may be either single-stranded or double-stranded. Single-stranded nucleic acids are preferred because they lack complementary strands that compete for the oligonucleotide precursors during the hybridization step of the method of the invention.

The phrase “target nucleic acid sequence” in the context of the present invention refers to a sequence of nucleotides to be measured, usually existing within a portion or all of a polynucleotide. In the present invention the identity of the target nucleotide sequence is usually known. The identity of the target nucleotide sequence may be known to an extent sufficient to allow preparation of various sequences hybridizable with the target nucleotide sequence and of oligonucleotides, such as probes and primers, and other molecules necessary for conducting methods in accordance with the present invention and so forth.

The target sequence usually contains from about 30 to 5,000 or more nucleotides, preferably 50 to 1,000 nucleotides. The target nucleotide sequence is generally a fraction of a larger molecule or it may be substantially the entire molecule such as a polynucleotide as described above. The minimum number of nucleotides in the target nucleotide sequence is selected to assure that the presence of a target polynucleotide in a sample is a specific indicator of the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g. transcription of a DNA template into RNA) and the efficiency of identification, detection, amplification, and/or other analysis of the target nucleotide sequence, where appropriate.

The term “oligonucleotide” refers to a polynucleotide, usually single stranded, usually a synthetic polynucleotide but may be a naturally occurring polynucleotide. The length of an oligonucleotide is generally governed by the particular role thereof, such as, for example, probe, primer, precursor and the like. Various techniques can be employed for preparing an oligonucleotide. Such oligonucleotides can be obtained by biological synthesis or by chemical synthesis. For short oligonucleotides (up to about 100 nucleotides), chemical synthesis will frequently be more economical as compared to the biological synthesis. In addition to economy, chemical synthesis provides a convenient way of incorporating low molecular weight compounds and/or modified bases during specific synthesis steps. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and- sample handling. Methods of oligonucleotide synthesis include phosphotriester and phosphodiester methods (Narang, et al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al., Proc. Nat. Acad. Sci. USA (1994) 91:5022-5026.

The phrase “oligonucleotide probe precursor” refers to a nucleic acid sequence that is complementary to a portion of the target nucleic acid sequence. The oligonucleotide precursor is a sequence of nucleoside monomers joined by phosphorus linkages (e.g., phosphodiester, alkyl and aryl-phosphate, phosphorothioate, phosphotriester), or non-phosphorus linkages (e.g., peptide, sulfamate and others). It may be a natural or synthetic molecule of single-stranded DNA and single-stranded RNA with circular, branched or linear shape and optionally including domains capable of forming stable secondary structures (e.g., stem-and-loop and loop-stem-loop structures). The oligonucleotide probe precursor contains a 3′-end and a 5′-end. An oligonucleotide probe precursor may comprise one or more modified nucleotides in accordance with the present invention and accordingly may be referred to as mass modified.

The term “mixture” refers to a physical mixture of two or more substances.

The phrase “oligonucleotide probe” refers to an oligonucleotide employed to bind to a portion of a polynucleotide such as another oligonucleotide or a target nucleic acid sequence. The design and preparation of the oligonucleotide probes are generally dependent upon the sequence to which they bind. The oligonucleotide probe precursors are a subset of oligonucleotide probes.

The phrase “oligonucleotide primer(s)” refers to an oligonucleotide that is usually employed in a chain extension on a polynucleotide template such as in, for example, an amplification of a nucleic acid, e.g., PCR. The oligonucleotide primer is usually a synthetic nucleotide that is single stranded, containing a sequence at its 3′-end that is capable of hybridizing with a defined sequence of the target polynucleotide. Normally, an oligonucleotide primer has at least 80%, preferably 90%, more preferably 95%, most preferably 100%, complementarity to a defined sequence or primer binding site. The number of nucleotides in the hybridizable sequence of an oligonucleotide primer should be such that stringency conditions used to hybridize the oligonucleotide primer will prevent excessive random non-specific hybridization. In the present invention the oligonucleotide probe precursor is an “oligonucleotide primer” when polymerase extension is employed.

The phrase “nucleoside triphosphates” refers to nucleosides having a 5′-triphosphate substituent. The nucleosides are pentose sugar derivatives of nitrogenous bases of either purine or pyrimidine derivation, covalently bonded to the 1′-carbon of the pentose sugar, which is usually a deoxyribose or a ribose. The purine bases include adenine (A), guanine (G), inosine (I), and derivatives and analogs thereof. The pyrimidine bases include cytosine (C), thymine (T), uracil (U), and derivatives and analogs thereof. Nucleoside triphosphates include deoxyribonucleoside triphosphates such as the four common deoxyribonucleoside triphosphates dATP, dCTP, dGTP and dTTP and ribonucleoside triphosphates such as the four common triphosphates rATP, rCTP, rGTP and rUTP. The term “nucleoside triphosphates” also includes derivatives and analogs thereof, which are exemplified by those derivatives that are recognized and polymerized in a similar manner to the underivatized nucleoside triphosphates.

The term “nucleotide” or “nucleotide base” or “base” refers to a base-sugar-phosphate combination that is the monomeric unit of nucleic acid polymers, i.e., DNA and RNA. The term as used herein includes modified nucleotides as defined below. In general, the term refers to any compound containing a cyclic furanoside-type sugar (β-D-ribose in RNA and β-D-2′-deoxyribose in DNA), which is phosphorylated at the 5′ position and has either a purine or pyrimidine-type base attached at the C-1′ sugar position via a β-glycosyl C1′-N linkage. These terms are interchangeable and will be denoted by a b. The nucleotide may be natural or synthetic, including a nucleotide that has been mass-modified including, inter alia, nucleotides having modified nucleosides with modified bases (e.g., 5-methyl cytosine) and modified sugar groups (e.g., 2′-O-methyl ribosyl, 2′-O-methoxyethyl ribosyl, 2′-fluororibosyl, 2′-amino ribosyl, and the like).

The term “DNA” refers to deoxyribonucleic acid.

The term “RNA” refers to ribonucleic acid.

The term “cDNA” refers to the complements of mRNA sequences (cDNA or cRNA).

The term “natural nucleotide” refers to those nucleotides that form the fundamental building blocks of cellular DNA, which are defined to include deoxycytidylic acid (pdC), deoxyadenylic acid (pdA), deoxyguanylic acid (pdG) and deoxythymidylic acid (pdT) and the fundamental building blocks of cellular RNA which are defined to include deoxycytidylic acid (pdC), deoxyadenylic acid (pdA), deoxyguanylic acid (pdG) and deoxyuridylic acid (pdU). pdU is considered to be a natural equivalent of pdT.

The term “natural nucleotide base” refers to purine- and pyrimidine-type bases found in cellular DNA and include cytosine (C), adenine (A), guanine (G) and thymine (T) and in cellular RNA and include cytosine (C), adenine (A), guanine (G) and uracil (U). U is considered a natural equivalent of T.

The phrase “modified nucleotide” refers to a unit in a nucleic acid polymer that contains a modified base, sugar or phosphate group. The modified nucleotide can be produced by a chemical modification of the nucleotide either as part of the nucleic acid polymer or prior to the incorporation of the modified nucleotide into the nucleic acid polymer. For example, the methods mentioned above for the synthesis of an oligonucleotide may be employed. In another approach a modified nucleotide can be produced by incorporating a modified nucleoside triphosphate into the polymer chain during an amplification reaction. Examples of modified nucleotides, by way of illustration and not limitation, include dideoxynucleotides, derivatives or analogs that are biotinylated, amine modified, alkylated, fluorophor-labeled, and the like and also include phosphorothioate, phosphite, ring atom modified derivatives, and so forth.

The phrase “Watson-Crick base pairing” refers to the hydrogen bonding between two bases, with specific patterns of hydrogen bond donors and acceptors having the standard geometries defined in “Principles of Nucleic Acid Structure”; Wolfram Saenger, Springer-Verlag, Berlin (1984).

The phrase “natural complement of a nucleotide” refers to the natural nucleotide with which a nucleotide most favorably forms a base pair according to the Watson-Crick base pairing rules. If the nucleotide can base pair with equal affinity with more than one natural nucleotide, or most favorably pairs with different natural nucleotides in different environments, then the nucleotide is considered to have multiple natural nucleotide complements.

The phrase “natural equivalent of a nucleotide” refers to the natural complement of the natural complement of the nucleotide. In cases where a nucleotide has multiple natural complements, then it is considered to have multiple natural equivalents.

The phrase “natural equivalent of an oligonucleotide probe precursor” refers to an oligonucleotide precursor in which each nucleotide has been replaced with its natural nucleotide equivalent. In cases where one or more of the original nucleotides has multiple natural equivalents, then the oligonucleotide precursors will be considered to have multiple natural equivalents, with the equivalents being chosen from all of the possible combinations of replacements.

The term “nucleoside” refers to a base-sugar combination or a nucleotide lacking a phosphate moiety.

The term “chain-terminating nucleoside triphosphate” refers a nucleoside triphosphate that is capable of being added to an oligonucleotide probe precursor in a chain extension reaction but is incapable of undergoing chain extension. Examples by way of illustration and not limitation include the four standard dideoxynucleotide triphosphates, mass-modified dideoxynucleotide triphosphate analogues, thio analogs of natural and mass-modified dideoxynucleotide triphosphates, arabanose, 3′-amino, 3′-azido, 3′-fluoro derivatives and the like.

The phrase “dideoxynucleoside triphosphate” refers to and includes the four natural dideoxynucleoside triphosphates (ddATP, ddGTP, ddCTP and ddTTP for DNA and ddATP, ddGTP, ddCTP and ddUTP for RNA) and mass-modified dideoxynucleoside triphosphates. The term may be denoted by ddNTP.

The phrase “extension nucleoside triphosphates” refers to and includes natural deoxynucleoside triphosphates, modified deoxynucleotide triphosphates, mass-modified deoxynucleoside triphosphates, 5′(α)-phosphothioate, and 5′-N (α-phosphoramidate) analogs of natural and mass-modified deoxy and ribonucleoside triphosphates and the like, such as those disclosed in U.S. Pat. No. 5,171,534 and U.S. Pat. No. 5,547,835, the relevant portions of which are incorporated herein by reference.

The phrase “nucleotide polymerase” refers to a catalyst, usually an enzyme, for forming an extension of a polynucleotide along a DNA or RNA template where the extension is complementary thereto. The nucleotide polymerase is a template dependent polynucleotide polymerase and utilizes nucleoside triphosphates as building blocks for extending the 3′-end of a polynucleotide to provide a sequence complementary with the polynucleotide template. Usually, the catalysts are enzymes, such as DNA polymerases, for example, prokaryotic DNA polymerase (I, II, or III), T4 DNA polymerase, T7 DNA polymerase, E. coli DNA polymerase (Klenow fragment, 3′-5′ exo-), reverse transcriptase, Vent DNA polymerase, Pfu DNA polymerase, Taq DNA polymerase, Bst DNA polymerase, and the like, or RNA polymerases, such as T3 and T7 RNA polymerases. Polymerase enzymes may be derived from any source such as cells, bacteria such as E. coli, plants, animals, virus, thermophilic bacteria, and so forth.

The term “amplification” of nucleic acids or polynucleotides refers to a method that results in the formation of one or more copies of a nucleic acid or polynucleotide molecule (exponential amplification) or in the formation of one or more copies of only the complement of a nucleic acid or polynucleotide molecule (linear amplification). Methods of amplification include the polymerase chain reaction (PCR) based on repeated cycles of denaturation, oligonucleotide primer annealing, and primer extension by thermophilic template dependent polynucleotide polymerase, resulting in the exponential increase in copies of the desired sequence of the polynucleotide analyte flanked by the primers. The two different PCR primers, which anneal to opposite strands of the DNA, are positioned so that the polymerase catalyzed extension product of one primer can serve as a template strand for the other, leading to the accumulation of a discrete double stranded fragment whose length is defined by the distance between the 5′ ends of the oligonucleotide primers. The reagents for conducting such an amplification include oligonucleotide primers, a nucleotide polymerase and nucleoside triphosphates such as, e.g., deoxyadenosine triphosphate (dATP), deoxyguanosine triphosphate (dGTP), deoxycytidine triphosphate (dCTP) and deoxythymidine triphosphate (dTTP). Other methods for amplification include amplification of a single stranded polynucleotide using a single oligonucleotide primer, the ligase chain reaction (LCR), the nucleic acid sequence based amplification (NASBA), the Q-beta-replicase method, and 3SR.

The terms “hybridization (hybridizing)” and “binding” in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like.

The term “complementary,” “complement,” or “complementary nucleic acid sequence” refers to the nucleic acid strand that is related to the base sequence in another nucleic acid strand by the Watson-Crick base-pairing rules. In general, two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G/U or U/G basepairs.

The term “hybrid” refers to a double-stranded nucleic acid molecule formed by hydrogen bonding between complementary nucleotides. The term “hybridize” refers to the process by which single strands of nucleic acid sequences form double-helical segments through hydrogen bonding between complementary nucleotides.

The term “mass-modified” refers to a nucleic acid sequence or single nucleotide whose mass has been changed either by an internal change, i.e., by addition, deletion, or substitution of a chemical moiety, to its chemical structure or by an external change, i.e., by the addition of a chemical moiety (atom or molecule) attached covalently, to its chemical structure. The chemical moiety is therefore referred to as a mass-modifying moiety.

The phrase “direct mass spectral analysis” refers to a method of mass spectral analysis that analyzes either the target nucleic acid sequence itself or the complement of the target nucleic acid sequence. The target nucleic acid sequence itself or its complement may be mass modified, contain additional nucleotide bases or be otherwise modified, provided that the target nucleic acid sequence or its complement is actually mass analyzed. However, the phrase does not include mass spectral analysis wherein a mass tag moiety that is indicative of the presence of target nucleic acid sequence is analyzed, such as those indirect methods described in PCT Application WO 95/04160.

The term “support” or “surface” refers to a porous or non-porous water insoluble material. The surface can have any one of a number of shapes, such as strip, plate, disk, rod, particle, including bead, and the like. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. Natural or synthetic assemblies such as liposomes, phospholipid vesicles, and cells can also be employed. Binding of oligonucleotides to a support or surface may be accomplished by well-known techniques, commonly available in the literature. See, for example, A. C. Pease, et al., Proc. Nat. Acad. Sci. USA, 91:5022-5026 (1994).

General Comments

The invention provides methods for profiling expression levels of a priori known target nucleic acid sequences in a complex mixture. The method comprises hybridizing oligonucleotide probe precursors to respective target nucleic acid sequences, either in solution or on a surface, subjecting the hybrids to a target mediated enzymatic reaction to form oligonucleotide products each having a unique mass, denaturing the hybrids and analyzing the oligonucleotide products by mass spectroscopy.

The present invention employs designed oligonucleotide probe precursors that are mass differentiated by using mass modified nucleotides. The current invention bypasses the need for mass tags. It employs determination of gene expression profiles from direct mass spectrometric analysis of altered oligonucleotide probe precursors is made possible. Furthermore, multiplexing schemes are designed so as to maximize the number of genes of interest that can be interrogated in a single assay, given all relevant constraints. When mass-tag technologies of the prior art are employed, the extent of multiplexing is severely restricted by the number of well-behaved, distinguishable mass tags. Moreover, in the present methods registration of the hybridization event is realized by an enzymatic or other reaction to form oligonucleotide products distinguishable by mass. As a result, some of the difficulties of non-specific hybridization, incurred when washing is used for the same purpose, are avoided.

The current invention provides advantages over the methods of the prior art discussed above. The methods of the present invention are highly parallel without the disadvantages associated with labeling. Enzymatic reactions in a polymerase extension approach (also in the ligase based approach) serve to proofread transient hybridization and the final reading step is carried out by mass spectrometry, which provides for quick analysis. As a result of the present invention a high multiplexing rate and a high throughput are achieved. In addition, reactions are carried out in solution phase followed by mass modification. Accordingly, it is the mass difference that provides the distinction of the hybridized probes and no washing separation step is necessary.

The present invention may be applied to a wide variety of assays such as, for example, expression based diagnostic assays including, e.g., cancer treatment determination assays and earlier detection assays where quantitative profiling of 10 to 200 preferably, 30 to 100, genes is key to the result. The present invention may be applied to association studies where quantitative genotyping is important.

Reagents of the Invention

Oligonucleotide Probe Precursors

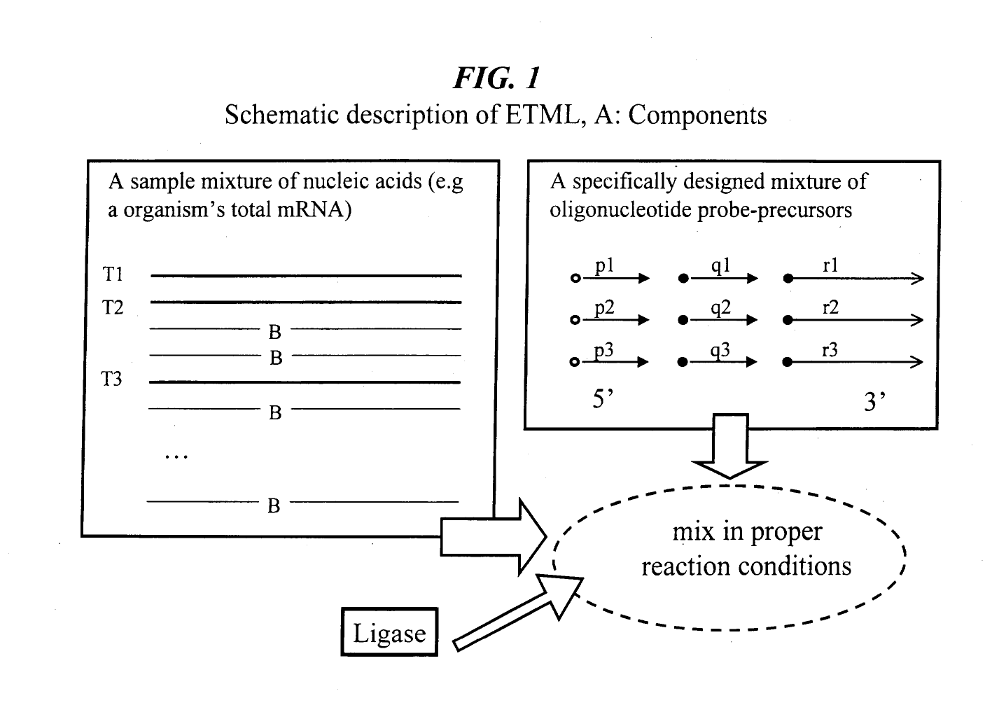

The oligonucleotide probe precursors useful in the method of the invention have a length of at least about 3 nucleotide units. Preferably, the oligonucleotide probe precursors have a length of at least about 4 nucleotide units, usually, at least about 5 nucleotide units. The length of the oligonucleotide probe precursors is dependent on the. type of processing involved. For example, when the processing comprises a target sequence mediated enzymatic extension approach, the length of the oligonucleotide probe precursors is about 12 to about 30 nucleotides, usually, about 15 to about 20 nucleotides. Alternatively, when the processing comprises ligation, the length of the oligonucleotide probe precursors depends on the number of such precursors employed for each target nucleic acid sequence. For example, when two oligonucleotide probe precursors are employed, the length of the precursors is about 8 to about 13 nucleotides, usually, about 9 to about 11 nucleotides. When three oligonucleotide probe precursors are employed, the length of the precursors is about 3 to about 13 nucleotides, usually, about 5 to about 13 nucleotides. It is preferred that the oligonucleotide probe precursors be of a length sufficient to serve as good substrates for ligation by the ligase yet not too long to serve as templates for ligation of complementary oligonucleotide probe precursors within the reaction mixture. The length of the oligonucleotide probe precursor may be selected independently for each oligonucleotide probe precursor in the mixture. Thus, it is possible to have a single mixture of oligonucleotide probe precursors having lengths of 3, 4, 5, 6 or more nucleotides. In a particular example, in a ligation based assay the lengths of the precursors p1, q1 and r1 may be 5, 5 and 13, respectively (see FIG. 1). Only the end of q1 and r1 designated with a bold dot can react enzymatically. This design provides most of the specificity by means of the 13-mer and more specificity and the registration by the additional 5-mers.

The oligonucleotide probe precursors useful in the method of the invention may each be represented by a single chemical species as opposed to being represented by a number of variants of similar chemical species, such as the ladder of reporter products used to represent the nucleotide sequence in the oligonucleotide described in PCT Application WO 95/04160 (Southern). Thus, each oligonucleotide probe precursor in the mixture of the invention possesses a single mass whereas each oligonucleotide in the mixture of WO 95/04160 is associated with a spectrum of masses, which represent the nucleotide sequence of interest as discussed above. It is important to recognize that the mass-tag approach disclosed by Southern utilizes cleavable mass tags in which only the tagged portion of the tagged oligonucleotides is analyzed in the mass spectrometer. As can be seen from the disclosure herein, this stands in contrast to the present invention, which relies on generating mass spectra of the oligonucleotide products themselves resulting from a target mediated process. Moreover, the mixture of mass-modified oligonucleotide probe precursors in the present invention is designed such that any given oligonucleotide sequence possesses only a single mass, that is, represents a single chemical species. This in not the case in the mass-tag approach disclosed by Southern. Due to the “ladder tag” design of the Southern approach, each discrete oligonucleotide sequence within the mixture is associated with a “spectrum” of mass entities.

To be useful in the methods of the present invention for determining amounts of particular target nucleic acid sequences, it is necessary to know which oligonucleotide probe precursors are present in the mixture. However, it is not absolutely necessary to know the amount of each oligonucleotide probe precursor. With this said, however, it is advantageous, to be able to control the concentration of each oligonucleotide probe precursor in the mixture to compensate for differences in duplex thermostabilities (see discussion below).

In one preferred embodiment, the oligonucleotide probe precursors are composed of both natural and mass-modified nucleotides. The identity and location of mass-modified nucleotides within the oligonucleotide probe precursors will depend upon a number of factors. These include the desired thermodynamic properties of the oligonucleotide probe precursor, the ability of an enzyme or set of enzymes (i.e. polymerases and ligases) to accommodate mass-modified nucleotides within the oligonucleotide probe precursor, and the constraints imposed by the particular synthesis method of the oligonucleotide probe precursors.

The oligonucleotide probe precursors may be mass modified either by an internal change, i.e., by addition, deletion, or substitution of a chemical moiety, to its chemical structure or by an external change, i.e., by the addition of a chemical moiety (atom or molecule) attached covalently, to its chemical structure. An oligonucleotide probe precursor may have both an internal change and an external change, more than one internal change, more than one external change or some combination thereof.

Suitable internal mass modifications include at least one chemical modification to the internucleoside linkage, sugar backbone or nucleoside base of the oligonucleotide probe precursor. Examples of suitable internally mass-modified X-mer precursors, by way of illustration and not limitation, are those that include 2′-deoxy-5-methylcytidine, 2′-deoxy-5-fluorocytidine, 2′-deoxy-5-iodocytidine, 2′-deoxy-5-fluorouridine, 2′-deoxy-5-iodo-uridine, 2′-O-methyl-5-fluorouridine, 2′-deoxy-5-iodouridine, 2′-deoxy-5(1-propynyl)uridine, 2′-O-methyl-5(1-propynyl)uridine, 2-thiothymidine, 4-thiothymidine, 2′-deoxy-5(1-propynyl)cytidine, 2′-O-methyl-5(1-propynyl)cytidine, 2′-O-methyladenosine, 2′-deoxy-2,6-diaminopurine, 2′-O-methyl-2,6-diaminopurine, 2′-deoxy-7-deazadenosine, 2′-deoxy-6methyladenosine, 2′-deoxy-8-'oxoadenosine, 2′-O-methylguanosine, 2′-deoxy-7-deazaguanosine, 2′-deoxy-8-oxoguanosine, 2′-deoxyinosine or the like.

Suitable external mass modifications include mass-modifying moieties linked to the oligonucleotide probe precursors. External mass-modifying moieties may be attached to the 5′-end of the probe precursor, to the nucleotide base (or bases), to the phosphate backbone, to the 2′-position of the nucleoside (nucleosides), to the terminal 3′-position and the like. Suitable external mass-modifying moieties include, for example, a halogen, an azido, sulfur, silver, gold, platinum, mercury, mass moieties of the type, W-R, wherein W is a linking group and R is a mass-modifying moiety and the like.

The linking group is involved in the covalent linkage between molecules W and R. The linking group will vary depending upon the nature of the molecules. Functional groups that are normally present or are introduced on the molecules are employed for linking. The linking groups may vary from a bond to a chain of from 1 to 100 atoms, usually from about 1 to 60 atoms, preferably 1 to 40 atoms more preferably 1 to 20 atoms, each independently selected from the group normally consisting of carbon, hydrogen, oxygen, sulfur, nitrogen, halogen and phosphorous. As a general rule, the length of a particular linking group can be selected arbitrarily to provide for convenience of synthesis and the incorporation of any desired group. The linking groups may be aliphatic or aromatic, although with diazo groups, aromatic groups will usually be involved. Common functionalities in forming a covalent bond between the linking group and the molecule to be conjugated are alkylamine, amidine, thioamide, ether, urea, thiourea, guanidine, azo, thioether and carboxylate, sulfonate, and phosphate esters, amides and thioesters. Usually, the linking group has a phosphate group, an amino group, alkylating agent such as halo or tosylalkyl, oxy (hydroxyl or the sulfur analog, mercapto) oxocarbonyl (e.g., aldehyde or ketone), or active olefin such as a vinyl sulfone or α-, β-unsaturated ester. These functionalities will be linked to amine groups, carboxyl groups, active olefins, alkylating agents, e.g., bromoacetyl.

Other suitable mass modifications should be obvious to those skilled in the art, including those disclosed in Oligonucleotides and Analogues, A Practical Approach, F. Eckstein (editor), IRL Press, Oxford, (1991); U.S. Pat. No. 5,605,798; and Japanese Patent No. 59-131909, which are incorporated herein by reference.

A primary goal of the invention is to generate oligonucleotide products each having a unique mass wherein the amount of each of the oligoriucleotide products is measured to determine the amount of a corresponding target nucleic acid sequence.

There will next be described three methods for synthesizing oligonucleotide probe precursors. This is by way of illustration and not limitation. Each of the methods described herein has certain advantages depending upon the degree of synthetic control over the individual oligonucleotides that is required. All three methods utilize standard phosphoramidite chemistries or enzymatic reactions that are known in the art.

The oligonucleotide probe precursors may be synthesized by conventional techniques, including methods employing phosphoramidite chemistry, including both 5′ to 3′ and 3′ to 5′ synthesis routes. Using an automated robotic workstation facilitates this process. This method allows for complete synthetic control of each oligonucleotide probe precursor with regard to composition and length. Individual synthesis also allows for QC analysis of each oligonucleotide probe precursor, which aids in final product manufacturing. Having individual samples of each oligonucleotide probe precursor also allows each oligonucleotide probe precursor to be present in the mixture at a specified concentration. This potentially may be helpful in compensating for different thermostabilities that may occur in the hybrids of the target nucleic acid sequence and the oligonucleotide probe precursor.

In another approach the mass-modified oligonucleotide probe precursors can be synthesized individually as described in the first method followed by a chemical modification of their 5′-termini with some type of mass modifier moiety. Only a small number of discrete mass modifiers are necessary in order to disperse the masses of resulting natural oligonucleotide mixture throughout the usable mass spectrometer mass range. This method is similar to that disclosed in U.S. Pat. No. 5,605,798, the relevant disclosure of which is incorporated herein by reference. It should be noted that, although the aforementioned patent describes a similar synthesis, it does not describe or suggest the use of mass-modified oligonucleotides for determining amounts of target nucleic acid sequences in complex mixtures thereof as described for the present invention.

The composition of the oligonucleotide probe precursors may influence the overall specificity and sensitivity of the assay. Moreover, having control over both their design and mode of synthesis allows for the incorporation of modifications that aid in their use in the methods of the invention. For example, the intemucleoside linkage on the phosphodiester backbone of the oligonucleotide probe precursors may be modified. In one embodiment, it is preferred that such chemical modification render the phosphodiester linkage resistant to nuclease digestion. Suitable modifications include incorporating non-bridging thiophosphate backbones, 5′-N-phosphoamidite intemucleotide linkages and the like.

The mass modification may increase the thermodynamic stability of the hybrids formed between the oligonucleotide probe precursor and target nucleic acid sequence to normalize the thermodynamic stability of the hybrids within the mixture. For example, 2,6-diaminopurine forms more stable base-pairs with thymidine than does adenosine. In addition, incorporating 2′-fluoro-thymidine increase the stability of A-T base pairs whereas incorporating 5-bromo and 5-methyl cytidine increases the stability of G-C base pairs.

The mass modification may decrease the thermodynamic stability of the hybrids formed between the oligonucleotide probe precursor and target nucleic acid sequence to normalize the thermodynamic stability of the hybrids within the mixture. A-T base pairs can be destabilized by incorporating 2′-amino-nucleosides. Inosine can also be used in place to guanosine to destabilized G-C base pairs. Incorporating N-4-ethyl-2′-deoxycytidine has been shown to decrease the stability of G-C base pairs. Incorporating the latter can normalize the stability of any given duplex sequence to an extent where its stability is made independent of A-T and G-C content (Nguyen et al., Nucleic Acids Res. 25, 3095 (1997)). Furthermore, the precursors may be mass-modified by a chemical modification at the 5′-terminus as long as the modification does not interfere with any subsequent reaction such as, for example, ligation or polymerase extension.

Modifications that reduce fragmentation of the oligonucleotide due to the ionization processes in mass spectrometry can also be introduced. For example, one approach is a 7-deaza modification of purines to stabilize the N-glycosidic bond and hence reduce fragmentation of oligonucleotides during the ionization process (see, for example, Schneider and Chait, Nucleic Acids Res v23, 1570 (1995)). Modification of the 2′ position of the ribose ring with an electron withdrawing group such as hydroxyl or fluoro may be employed to reduce fragmentation by stabilizing the N-glycosidic bond (see, for example, Tang, et al., J Am Soc Mass Spectrom, 8, 218-224, 1997).

In one embodiment of the present invention mass-modified dideoxynucleoside triphosphates may also possess an additional chemical component that increases the ionization efficiency of the desired extended oligonucleotide probe precursor relative to the unextended oligonucleotide probe precursors or any other undesirable components present in the sample mixture. Usually, the ionization efficiency is increased by at least a factor of 2, more usually by a factor of 4 and preferably by a factor of 10. Exemplary of such additional chemical components are primary amines, which can act as protonation sites and thus support single positive ion species for MALDI analysis (Tang et al., 1997, supra). It is also possible to incorporate quaternary amines, which possess a fixed positive charge. This class of chemical groups may be incorporated into non-cleavable mass-modified moieties using NHS ester chemistry similar to that disclosed by Gut, et al., in WO 96/27681 Briefly, the succinimide ester of a quaternary ammonium charged species, such as trimethylammonium hexyryl-N-hydroxysuccinimidyl ester is reacted with a nucleoside derivative having a primary aliphatic amino group. A suitable nucleoside is, for example, a known terminator such as the 3′-amino derivatives of the 2′-deoxynucleosides. Other suitable nucleosides would be the 5-[3-amino-1-propynyl]-pyrimidine and 7-deaza-[3-amino-1-propynyl]-purines derivatives similar to those used to generate the fluorescently labeled ddNTPs described by Prober, et al., (Science, 238, 336 (1987)).

Oligonucleotide probe precursors are designed in accordance with the nature of the target nucleic acid sequences. In general, the sequence information about the target nucleic acid sequences is known to the extent needed to design the oligonucleotide probe precursors. Other considerations in the design of the oligonucleotide probe precursors include the expected background message, the planned assay conditions and methods such as the nature of the enzymatic reaction used, and so forth. A more detailed discussion of the design of the oligonucleotide probe precursors is set forth below.

Methods of the Invention

General Description of the Methods

As mentioned above, one aspect of the present invention is a method of quantitatively analyzing a set of target nucleic acid sequences. A set of oligonucleotide probe precursors is hybridized to the target nucleic acid sequences to produce hybrids. The hybrids are processed to alter the mass of each of the oligonucleotide probes in the hybrids in a target sequence mediated reaction to produce products, each of which has a unique mass that is characteristic of its respective target nucleic acid and is not the result of the presence of a mass tag in the oligonucleotide product. An example of a target sequence mediated reaction by way of illustration and not limitation is an enzymatic assay such as, for example, a polymerase extension assay, a ligase assay, and the like. The products are analyzed by means of mass spectrometry and the results are related to the amount of each of the target nucleic acid sequences in the set. By the phrase “not the result of the presence of a mass tag” is meant that the intrinsic masses of reaction modified probes are employed for detection and quantification and not any external moieties.

In the method of the invention a set of oligonucleotide probe precursors prepared as described above is combined with the target nucleic acid sequences in the mixture to be studied. The combination is subjected to hybridization conditions to form hybrids between appropriate oligonucleotide probe precursors and respective target nucleic acid sequences. The hybrids are processed to alter the mass of the oligonucleotide probe precursor portions of the hybrids as described herein. This alteration may be accomplished either by an enzymatic or chemical reaction. Suitable enzymatic techniques include polymerase extension, ligation, and the like. Suitable chemical techniques include condensation of activated oligonucleotide probe precursors using carbodiimides and cyanogen bromide derivatives and the like. The following discussion is a brief description of some of the various processes; a more detailed discussion is set forth below. For enzymatic target mediated extension (ETME), hybridized oligonucleotide probe precursors are extended by polymerizing a single nucleotide at the 3′-end of the hybridized oligonucleotide probe precursors using a nucleotide polymerase. For the enzymatic target mediated ligation (ETML), adjacent hybridized oligonucleotide probe precursors are ligated together prior to analysis using a ligase. It should be noted that, although it is preferable that all of the adjacent hybridized oligonucleotide probe precursors are ligated, it is not a requirement. In other words, it is not necessary to have a 100% efficient reaction.

Detailed Description of the Methods

The following description is directed to methods and reagents for measuring the amount of each target nucleic acid sequence present in a set of such sequences. An example of such a method is gene expression profiling. The methods and reagents utilize oligonucleotide probe precursors and enzymatic or chemical processes to alter the length, and concomitantly the mass, of only those oligonucleotide probe precursors within a defined mixture that are complementary to, and hybridized to, the target nucleic acid sequences. The following description is by way of illustration and not limitation.

A medium is prepared comprising the target nucleic acid sequences. This usually involves obtaining a sample such as a cell sample, which provides the set of target nucleic acid sequences such as a set of genes. The sample may or may not be processed prior to use. The medium is then mixed with the designed oligonucleotide probe precursors and an enzymatic or chemical reaction is carried out to alter the mass of the hybridized oligonucleotide probe precursors. The oligonucleotide product mixture of the reaction is then analyzed by mass spectrometry. Such analysis may involve generation of raw data output followed by computerized data analysis to obtain the output in the form of quantitative results for the target nucleic acid sequences in the set. Thus, for example, expression levels for all genes in a set of genes of interest may be obtained with appropriate indications of confidence levels.

In ETME the combination of reagents is subjected to conditions under which the oligonucleotide probe precursors hybridize to respective target nucleic acid sequences and are extended by one nucleotide in the presence of a chain-terminating nucleoside triphosphate that is complementary to nucleotide of the target nucleic acid sequence adjacent to the hybridized oligonucleotide probe precursor. Generally, an aqueous medium is employed. Other polar cosolvents may also be employed, usually oxygenated organic solvents of from 1-6, more usually from 1-4, carbon atoms, including alcohols, ethers and the like. Usually these cosolvents, if used, are present in less than about 70 weight percent, more usually in less than about 30 weight percent.

The pH for the medium is usually in the range of about 4.5 to 9.5, more usually in the range of about 5.5 to 8.5, and preferably in the range of about 6 to 8. Various buffers may be used to achieve the desired pH and maintain the pH during the determination. Illustrative buffers include borate, phosphate, carbonate, Tris, barbital and the like. The particular buffer employed is not critical to this invention but in individual methods one buffer may be preferred over another.

The reaction is conducted for a time sufficient to produce the extended oligonucleotide probe precursor to form an oligonucleotide product that comprises the oligonucleotide probe precursor plus one nucleotide from a chain terminating nucleoside triphosphate. Generally, the time period for conducting the entire method will be from about 10 to 200 minutes. It is usually desirable to minimize the time period.

The concentration of the nucleotide polymerase is usually determined empirically. Preferably, a concentration is used that is sufficient to extend most if not all of the oligonucleotide probe precursors that specifically hybridize to respective target nucleic acid sequences. The primary limiting factors are generally reaction time and cost of the reagent.

The number of the target nucleic acid molecules can be as low as 10⁷ in a sample but generally may vary from about 10⁸ to 10¹¹, more usually from about 10¹⁰ to 10¹² molecules in a sample, preferably at least 10⁻¹¹M in the sample and may be 10⁻¹⁰ to 10⁻⁷M, more usually 10⁻⁸ to 10⁻⁶M. In general, the reagents for the reaction are provided in amounts to achieve extension of the hybridized oligonucleotide probe precursors. The number of each oligonucleotide probe precursor molecules is generally 10¹⁰ and is usually about 10¹⁰ to about 10¹¹, preferably, about 10¹⁰ to about 10¹² for a sample size that is about 10 microliters. The concentration of each oligonucleotide probe precursor may be adjusted according to its thermostability as discussed above. The absolute ratio of target nucleic acid to oligonucleotide probe precursor is to be determined empirically. The concentration of the chain-terminating nucleoside triphosphates in the medium can vary depending upon the affinity of the nucleoside triphosphates for the polymerase. Preferably, these reagents are present in an excess amount. The nucleoside triphosphates are usually present in about 10⁻⁷ to about 10⁻⁴ M, preferably, about 10⁻⁶ to about 10⁻⁴ M.

The reaction temperature can be in the range of from about 0° C. to about 95° C. depending upon the type of polymerase used, the concentrations of target nucleic acid and oligonucleotide probe precursors and the thermodynamic properties of the oligonucleotide probe precursors in the mixture. For example, at 40 nM target nucleic acid sequence, 40 nM 6-mer, and 7 nM Bst Polymerase, between 20% and 50% of the 6-mer can be extended at 5° C. in 2 hours depending upon the sequence of the 6-mer. Similar extension efficiencies are obtained at 20° C. indicating that the extension efficiency is not solely dependent upon the thermodynamics of the X-mer/target interaction. Importantly, it may be beneficial to cycle the incubation temperature. Cycling could help to expose structured region of the target nucleic acid sequence for oligonucleotide probe precursor binding and subsequent extension as well as facilitate turnover of the extension products. Thus, the overall sensitivity of ETME could be markedly increased by allowing a given target molecule to act as a template for multiple oligonucleotide probe precursor binding and subsequent extension reactions. In accordance with this aspect of the invention, one cycle may be carried out at a temperature of about 75° C. to about 95° C. for about 0.1 to 5 minutes, more usually about 0.5 to 2 minutes and another cycle may be carried out at a temperature of about 5° C. to about 45° C. for about 1 to 20 minutes, more usually about 5 to 15 minutes. The number of cycles may be from about 2 to about 20 or more. In general, the cycle temperatures and duration are selected to provide optimization of the extension of the hybridized oligonucleotide probe precursor of given length.

The order of combining of the various reagents to form the combination may vary. Usually, the sample containing the target nucleic acid sequences is combined with a pre-prepared combination of chain-terminating nucleoside triphosphates and nucleotide polymerase. The oligonucleotide probe precursors may be included in the prepared combination or may be added subsequently. However, simultaneous addition of all of the above, as well as other step-wise or sequential orders of addition, may be employed provided that all of the reagents described above are combined prior to the start of the reactions.

As mentioned above, in ETME an assay is performed wherein a designed mixture of oligonucleotide probe precursors is allowed to hybridize to sequences in the set of target nucleic acid sequences. Then, target mediated extension takes place. An example of such a method is outlined next. A (dideoxy) nucleotide that extends an oligonucleotide probe precursor p in this reaction is designated as e(p). Usually, the design process for the oligonucleotide precursors is conducted in such a way as to identify just one such precursor. However, it is within the purview of the present invention to use more than one oligonucleotide probe precursor per target nucleic acid sequence, for example, about 2 to about 5 such precursors per target nucleic acid sequence. A set of genes of interest is designated g₁, g₂, . . . , gₛ. A set of oligonucleotide probe precursors is identified. For example, the set may comprise oligonucleotide probe precursors of length about 15 to about 30 nucleotides, preferably, about 17 to about 23 nucleotides. The length of the oligonucleotide precursors is dependent on such factors as sequence specificity, and mass diversity. The oligonucleotide precursors generally have the following properties as illustrated, by way of example and not limitation, with one set of 20-mers p₁, p₂, . . . , pₛ with the following properties:

1. For every 1≦i≦s, pᵢ is a Watson-Crick complement of some substring of gᵢ. (Preferably this substring represents a non-structured region of gᵢ's mRNA).

2. For all pairs of oligonucleotide probes pᵢ, pⱼ, the longest 3′-end of pᵢ that is reverse complementary to any substring of pⱼ, is no more than 5, preferably, no more than 3 nucleotides long.

3. The masses m(pᵢ)+m(e(pᵢ)) are all different.

4. None of the probes pᵢ has a close match complement in the total mRNA pool of the organism of interest except, of course, the nucleic acid sequence of gᵢ.

The following assay is performed:

1. Sample comprising the target nucleic acid sequences is mixed with a mixture containing the oligonucleotide probe precursors p₁, p₂, . . . , pₛ.

2. The precursors are allowed to hybridize to the target nucleic acid sequences. The reaction mixture includes chain terminating nucleotides and polymerase. The oligonucleotide probe precursors that are hybridized to their respective target nucleic acid sequence are extended by one nucleotide. The quantities of the resulting extended products depend on the quantity of mRNA expressed by the respective target genes.

3. Following denaturing of duplexes the mixture is analyzed by mass spectroscopy.

The resulting mass spectrum is then analyzed to obtain the expression profile of the genes g₁, g₂, . . . , gₛ. To each gene gᵢ there is a corresponding extended product of a unique mass produced in a one to one manner because of the aforementioned properties of the design herein. More explicitly, this mass is mᵢ=m(pᵢ)+m(e(pᵢ))−18 amu. The one to one relationship is achieved because of the properties that the masses m(pᵢ)+m(e(pᵢ)) are all different and none of the probes pᵢ has a close match complement in the total mRNA pool of the organism of interest except, of course, the nucleic acid sequence of gᵢ.

1. To determine the amount, e.g., expression levels, of gᵢ the intensity of the peak at mᵢ is measured. Contribution to this peak can only come from gᵢ-mediated extension of pᵢ by e(pᵢ) because of the above design criteria for the oligonucleotide probe precursors.

2. The intensity of the peak is linear in (or indicative of) the quantity of the corresponding oligonucleotide.

Mathematical analysis and simulations show that, when s˜500-1000, all conditions stated above, regarding the relationship of the probes/precursors to themselves, their target genes and the background message can be satisfied, assuming that each target mRNA has at least 4-6 sensitive probe candidates, and possibly using mass modified base analogues. The dynamic range assumptions were not demonstrated to any extent. Some relaxation of these can be accommodated, of course, yielding a less quantitative measurement.

ETML is another method for generating oligonucleotide products based on the presence and amount of target nucleic acid sequences. Oligonucleotide probe precursors are allowed to hybridize to respective target nucleic acid sequences according to Watson-Crick base-pairing rules. The hybridized oligonucleotide probe precursors adjacent to one another are linked together, either enzymatically or chemically, in a target mediated reaction. Enzymatic ligation employs a ligase such as DNA ligase that assists in the formation of a phosphodiester bond to link two adjacent bases in separate oligonucleotides. Such ligases include, for example, T4 DNA ligase, Taq DNA Ligase, E. coli DNA Ligase and the like. Alternatively, adjacent oligonucleotide probe precursors may be ligated chemically using a condensing agent. Suitable condensing agents include, for example, carbodiimides, cyanogen bromide derivatives, and the like. The resulting ligated oligonucleotide products are analyzed by mass spectroscopy.

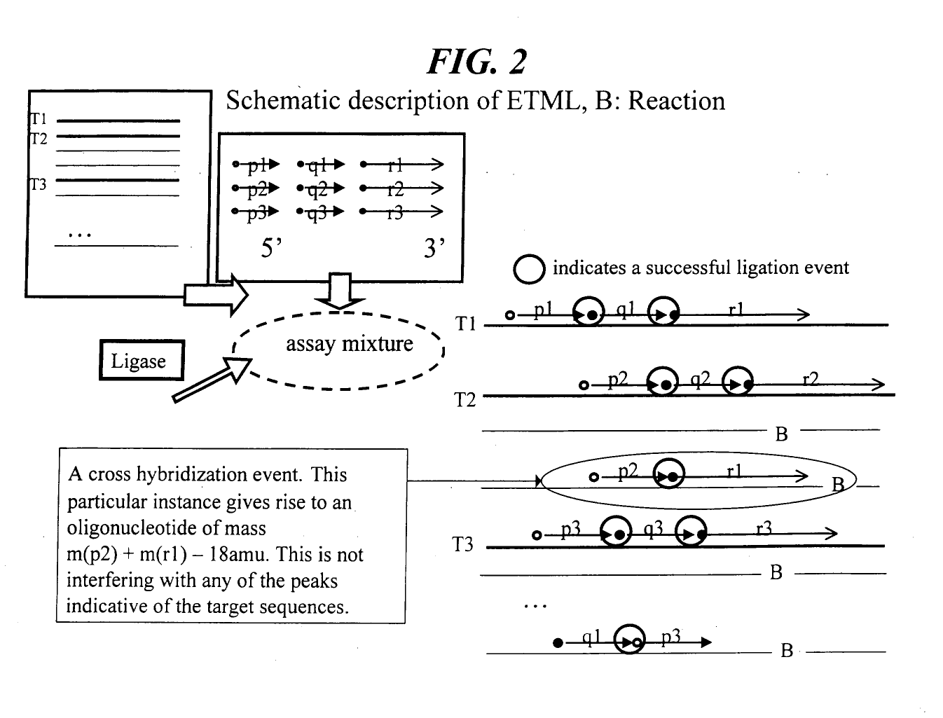

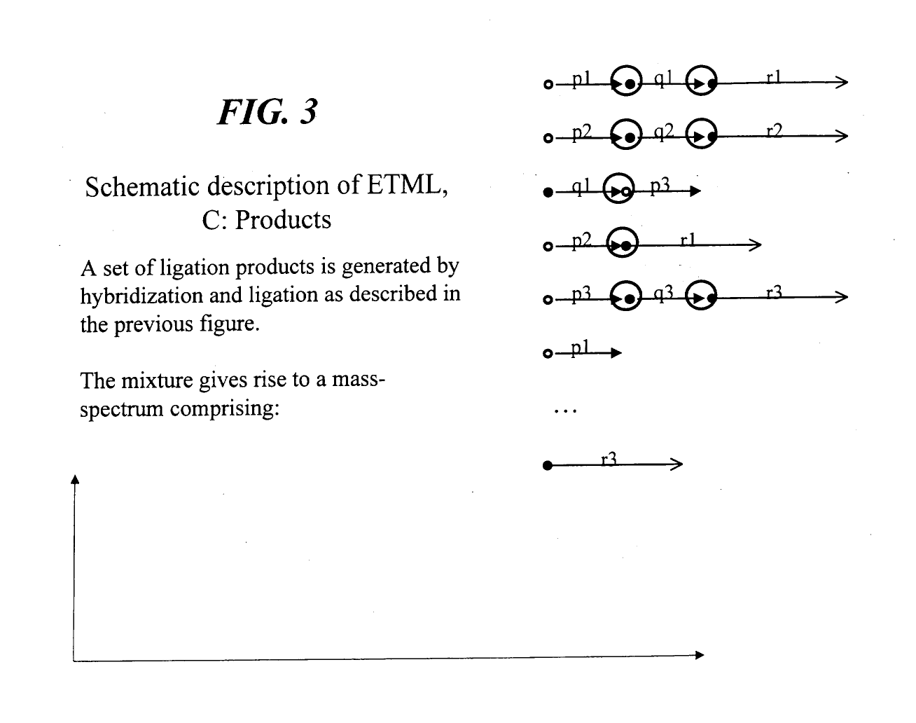

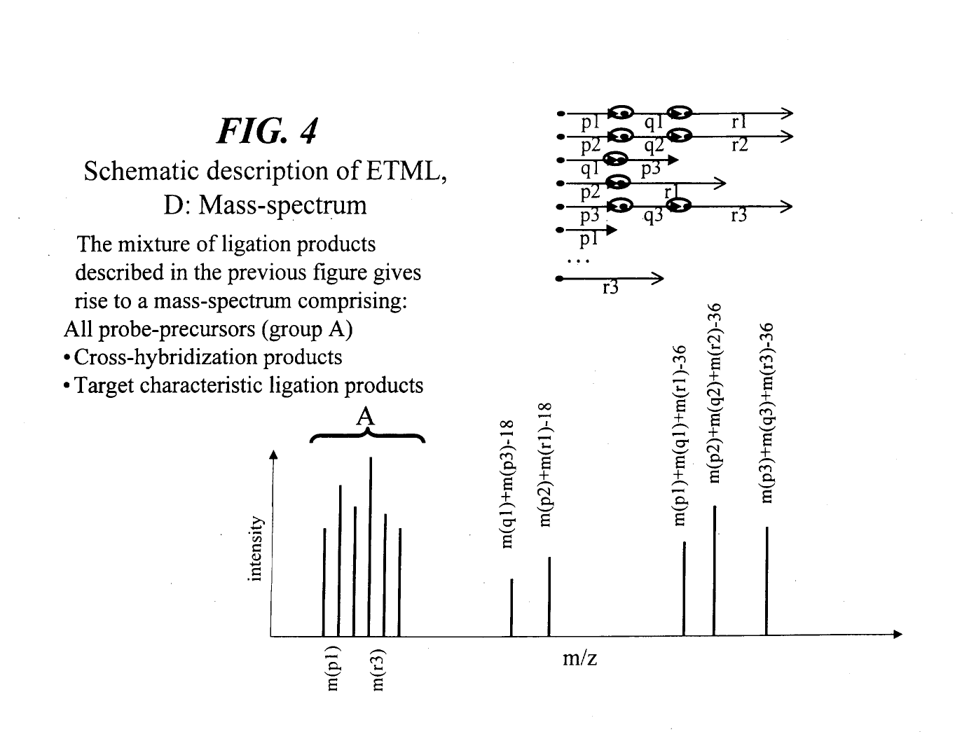

The above is illustrated in FIGS. 1-4 by way of illustration and not limitation. A sample containing a mixture of nucleic acids (T1, T2 and T3) is combined with oligonucleotide probe precursors p1, q1 and r1 for target T1, p2, q2 and r2 for target T2 and p3, q3 and r3 for target T3 (FIG. 1) in proper proportions and under proper reaction conditions, which include the presence of a ligase, and are allowed to hybridize to respective target nucleic acid sequences according to Watson-Crick base-pairing rules. Referring to FIG. 2, the hybridized oligonucleotide probe precursors adjacent to one another are linked together enzymatically in a target-mediated reaction to give product. In a variation of the above method the oligonucleotide probe precursors may be linked together chemically. Ligation events are indicated by O. As can be seen in FIG. 2, a cross-hybridization event is depicted between p2 and r1. However, the product of the cross-hybridization has a mass of m(p2)+m(r1)−18 amu, which does not interfere with any of the peaks indicative of the target sequences. Referring to FIGS. 3 and 4, a set of ligation products is generated by hybridization and ligation, which is subject to analysis by mass spectrometry. As can be seen with reference to FIG. 4, the mass spectrum comprises the probe precursors (group A), cross-hybridization products and products characteristic of target-mediated ligation.

The conditions for carrying out the reactions in this approach are similar to those described above. The pH for the medium is usually in the range of about 4.5 to 9.5, more usually in the range of about 5.5 to 8.5, and preferably in the range of about 6 to 8. The reaction is conducted for a time sufficient to produce the desired ligated product. Generally, the time period for conducting the entire method will be from about 10 to 200 minutes. It is usually desirable to minimize the time period. The reaction temperature can vary from 0° C. to 95° C. depending upon the type of ligase used, the concentrations of target and X-mers and the thermodynamic properties of the X-mers in the mixture. As in the case of ETME, it may be beneficial to cycle the incubation temperature to help expose structured region of the target for oligonucleotide probe precursor binding and subsequent ligation as well as to facilitate turnover of the ligated products.

The concentration of the ligase is usually determined empirically. Preferably, a concentration is used that is sufficient to ligate substantially all of the oligonucleotide probe precursors that specifically hybridize to the target nucleic acid sequences in order to obtain a quantitative result. The primary limiting factors are generally reaction time and cost of the reagent. The concentration of each oligonucleotide probe precursor is generally as described above for ETME and may be adjusted according to its thermostability as discussed above. The absolute ratio of target to oligonucleotide probe precursor is to be determined empirically.

In ETML a designed mixture of oligonucleotide probe precursors is allowed to hybridize to target nucleic acid sequences in a mixture; then, target mediated ligation takes place. The resulting product oligonucleotides are analyzed by mass spectrometry. Usually, the number of oligonucleotide probes involved in the ligation for each target nucleic acid sequence is about 2 to about 4, preferably, about 2 to 3. In an example by way of illustration and not limitation, a set of genes of interest g₁, g₂, . . . , gₛ is analyzed. The oligonucleotide precursors are about 3 to about 18 nucleotides, preferably, about 4 to about 15 nucleotides, in length. Desirably, the oligonucleotide probe precursors are short, i.e., about 4 to about 10 nucleotides in length to substantially reduce or eliminate any potential probe to probe priming problems. However, the rightmost precursor in a three-part ligation assay may be longer without risking precursor to precursor priming.

Probe to probe priming may be reduced further by the use of selective binding complementary nucleotide chemistry. In this approach, a matched pair of oligonucleotides is employed. Each member of the pair is complementary or substantially complementary in the Watson Crick sense to a target sequence of duplex nucleic acid where the two strands of the target sequence are themselves complementary to one another. The oligonucleotides in one member of the matched pair of oligonucleotides include modified bases of such nature that each of the modified bases forms a stable hydrogen bonded base pair with the natural complementary base but does not form a stable hydrogen bonded base pair with its modified partner in the other member of the pair of oligonucleotides. This is accomplished when in a hybridized structure the modified base is capable of forming two or more hydrogen bonds with its natural complementary base, but only one hydrogen bond with its modified partner. Due to the lack of stable hydrogen bonding with each other, the matched pair of oligonucleotides has a melting temperature under physiological or substantially physiological conditions of approximately 40° C. or less. However, each of the matched pair of oligonucleotides forms a substantially stable hybrid with the target sequence in each strand of the duplex nucleic acid. For a more detailed discussion of selective binding complementary nucleotide chemistry, see U.S. Pat. No. 5,912,340; Woo, J., et al., Nucleic Acids Research 24, 2470-2475 (1996) and Kutyavin I. V., et al., Biochemistry 35, 11170-11176 (1996).

For the particular example discussed below, three sets of oligonucleotide probe precursors, which are 6-7-mers, are employed, namely, p₁, p₂, . . . , pₛ, q₁, q₂, . . . , qₛ, r₁, r₂, . . . , rₛ. In general, the oligonucleotide probe precursors have the following properties:

1. For every 1 [symbol] i [symbol] s, the concatenated sequence, in this example pᵢq₁rᵢ, is a Watson-Crick complement of some subsequence (substring) of gᵢ. (Preferably, this substring represents a non-structured region of gᵢ's cDNA).

2. The masses of the oligonucleotide products, namely, m(pᵢ)+m(qᵢ)+m(rᵢ)−36, are all different.

3. Concatenated sequences, in this example being of the form pᵢqⱼrₖ or of the form Attorney Docket No. 10990147-1 qᵢqⱼrₖ or of the form pᵢqⱼqₖ or of the form qᵢqⱼqₖ, have no close match complements in the total mRNA pool of the organism of interest (except for the form pᵢqⱼrₖ when i=j=k, which is covered by 1 above). In fact, such close mismatches can be allowed providing that the mass of the spurious product is different from the mass of all desired products of the reaction. For example: m(pᵢ)+m(qⱼ)+m(rₖ) is different from all the numbers m(pᵢ)+m(qᵢ)+m(rᵢ), 1 [symbol] i [symbol] s.

Preferably, the oligonucleotide probe precursors that hybridize to respective target nucleic acid sequences at the farthest 3′ position of all oligonucleotide probe precursors, e.g., pᵢ s, can only ligate at their 5′-end. A modification that blocks ligation may be introduced at the 3′-terminus of the oligonucleotide precursors that are in this category. Blocking of the 3′-terminus may be accomplished, for example, by employing a group that cannot undergo condensation, such as, for example, an unnatural group such as a 3′-phosphate, a 3′-terminal dideoxy, a polymer or surface, or other means for inhibiting ligation. Also, the oligonucleotide probe precursors that hybridize to respective target nucleic acid sequences at the farthest 5′ position of all oligonucleotide probe precursors, e.g., rᵢ's preferably can only ligate at their 3′-end. A modification that blocks ligation may be introduced at the 5′-terminus of the oligonucleotide probe precursors that are in this category.

Blocking of the 5′-terminus may be accomplished, for example, by employing a group that cannot undergo extension through a ligation reaction, such as, for example, a natural 5′-hydroxyl group, an unnatural group such as a 5′-terminal methoxy group, a polymer or surface, or other means for inhibiting chain extension through ligation. Such an end group can be introduced at the 5′ end of the precursor during solid phase synthesis or a group can be introduced that can subsequently be modified. The details for carrying out the above modifications are well known in the art and will not be repeated here.

The level of phosphorylation of the 5'terminus of the oligonucleotide probe precursors can affect the extent of ligation (overall number of ligated products) and the length of ligation products. In the above example, the oligonucleotide probe precursors in the first set may possess, for example, a 5′ phosphorylated terminus and a 3′ blocked terminus (p--y) whereas the oligonucleotide probe precursors in the latter set may possess, for example, a 5′-blocked terminus and a 3′-hydroxyl terminus (y-p) and the third set of oligonucleotide probe precursors may possess both 5′ and 3′ hydroxyl termini (o--o). This results in only ligation products having the form y--p/o--o/p--y.

The following assay is performed:

1. The sample containing the target nucleic acid sequences is mixed with the sets oligonucleotide probe precursors, which were synthesized in accordance with the above considerations.

2. The oligonucleotide probe precursors are allowed to hybridize to the target nucleic acid sequences and ligase is introduced. The oligonucleotide probe precursors that are hybridized to their respective target nucleic acid sequences become ligated. The quantities of the resulting oligonucleotide products are linear in (or indicative on the quantity of mRNA expressed by the respective target genes.

3. The hybrids are denatured.

4. The denatured mixture is analyzed by mass spectroscopy.

The resulting mass-spectrum is then analyzed to obtain the expression profile of the genes g₁, g₂, . . . , gₛ as follows:

1. To each gene gᵢ corresponds a unique mass of an oligonucleotide product in a one to one manner. In the above example, this mass is mᵢ=m(pᵢ)+m(qᵢ)+m(rᵢ)−36 amu.

2. To determine the expression level of gᵢ the intensity of the peak at mᵢ is measured. Contribution to this peak can only come from gᵢ -mediated ligation of the oligonucleotide probe precursors, e.g., pᵢ, qᵢ and rᵢ, because of the design of the oligonucleotide probe precursors. It should be noted that probe mediated ligation is substantially negligible since the oligonucleotide probe precursors are short.

3. The peak intensity is linear in (or indicative of) the quantity of the corresponding oligonucleotide probe precursor, which in turn relates to the amount of the target nucleic acid sequence.

The above discussion is directed primarily to interrogating target nucleic acid sequences free in solution. However, it is also contemplated that the present methodology can be used in conjunction with surface-bound oligonucleotides such as arrays of oligonucleotides. The arrays generally involve a surface containing a mosaic of different oligonucleotides that are individually localized to discrete, known areas of the surface. Such ordered arrays containing a large number of oligonucleotides have been developed as tools for high throughput analyses of genotype and gene expression. Oligonucleotides synthesized on a solid support recognize uniquely complementary nucleic acids by hybridization, and arrays can be designed to identify specific target sequences, analyze gene expression patterns or identify specific allelic variations.

The present invention may be practiced using oligonucleotides attached to a support. In the present invention arrays of oligonucleotides such as DNA arrays can be generated such that the DNA probes are attached to the surface at their 3′ terminus through some type of photo- or chemically-cleavable linker. The linker may be cleavable by light, chemical, oxidation, reduction, acid-labile, base-labile, and enzymatic methods. These surface bound probes also have 5′ terminal phosphate. Exemplary of photo cleavable linkers are those based on the o-nitrobenzyl group such as those described in WO 95/04160 and so forth. Exemplary of linkers that are cleavable by reduction are those having a dithioate functionality that can be cleaved by mild reducing agents such as dithiothreitol or β-mercaptoethanol. Exemplary of acid labile cleavable linkers are those containing a 5′-N-phosphoamidite internucleotide linkage or an abasic nucleotide as a component of the linker, and so forth. Exemplary of base labile cleavable linkers are those containing a ribonucleotide as component of the linker and so forth.

For example, ETML may be carried out on an array prepared according to known techniques. Target nucleic acid sequences are allowed to hybridize to a generic (all k-mer) array prior to conducting a target-mediated ligation. Oligonucleotide probe precursors are allowed to hybridize to respective target nucleic acid sequences and adjacent hybridized precursors are ligated. The resulting product oligonucleotides are then analyzed by mass spectrometry. Each entry of the array is separately analyzed. The following example is by way of illustration and not limitation. The target nucleic acid sequences are a set of genes of interest g₁, g₂, . . . , gₛ. Two sets of about (6-7)-mers p₁, p₂, . . . , pₛ, q₁, q₂, . . . , qₛ with the following properties are employed:

1. For every 1 [symbol] i [symbol] s, the concatenated sequence pᵢqᵢ is a Watson-Crick complement of some substring of gᵢ. (Preferably this substring represents a non-structured region of g₁'s mRNA).

2. If for some k-mer u there are two genes gᵢ and gⱼ such that the concatenated sequences upᵢqᵢ and upⱼqⱼ are Watson-Crick complements of gᵢ and gⱼ respectively, then the masses m(pᵢ)+m(qᵢ) and m(pⱼ)+m(qⱼ) must be different.

3. Concatenated sequences of the form upᵢqⱼ where u is some k-mer have no close match complements in the total mRNA pool of the organism of interest (except when i=j which is covered by 1 above). In fact, such close mismatch can be allowed providing that m(pᵢ)+m(qⱼ) is different from all the numbers m(pᵢ)+m(qᵢ), where i ranges over all genes gᵢ such that for the fixed u under consideration upᵢqᵢ is a Watson-Crick complement of some substring of gᵢ.

The following assay is performed:

1. The target nucleic acid sequences are allowed to hybridize to an array of k-mers, which are attached to the surface by cleavable linkers.

2. A mixture containing the oligonucleotide precursors is next added to the array. The three sets, of oligonucleotide precursors are synthesized in accordance with the discussion above. The qᵢ s can only ligate at their 3′-end.

3. The oligonucleotide precursors are allowed to hybridize to the target nucleic acid sequences and a ligase is introduced. The oligonucleotide precursors that are hybridized to their respective targets become ligated. The quantities of the resulting oligonucleotide products are linear in (or indicative of) the quantity of mRNA expressed by the respective target genes.

4. Each entry in the array is analyzed entry by entry by cleaving the linker and analyzing the resulting mixture by mass spectroscopy.

The resulting mass-spectra are then analyzed to obtain the expression profile of the genes g₁, g₂, . . . , gₛ as follows:

1. To each gene gᵢ corresponds a unique mass of an oligonucleotide product, which is part of one of the spectra (pertaining to some k-mer u) in a one to one manner. More explicitly, this mass is mᵢ=m(pᵢ)+m(q₁)+m(u)−36 amu.

2. To determine the expression level of gᵢ, the intensity of the peak at m₁ is measured in the spectrum pertaining to u. Contribution to this peak can only come from gᵢ- mediated ligation of u, pᵢ, and qᵢ because of the design of the oligonucleotide precursors as discussed above. Probe mediated ligation is substantially negligible since the precursors are too short.

3. The intensity is linear in (or indicative of) the quantity of the corresponding oligonucleotide.

The array employed in the foregoing examples is an all k-mer array or a type of generic array. However, many other array formats may be employed. Accordingly, the foregoing and the following discussion are by way of illustration and not limitation. Another variant of a generic array includes, for example, an array containing a sub-set of all possible 7-mers. The solution precursors are then designed, for each target sequence, to hybridize immediately adjacent to a complement to one of the array members that is a subsequence of the target sequence. In another approach a custom designed array may be used such as in methods that use only hybridization arrays. The mass spectrometry step adds sensitivity and specificity.

ETME and ETML possess a number of desirable attributes. First, all are solution-based systems and are governed by standard solution mass-action and diffusion processes. This stands in contrast to unassisted surface-based array hybridization systems, where the probe is physically attached to the surface and unable to diffuse, thus slowing the kinetics of hybridization. In contrast to surface-bound arrays, it is a characteristic of the present invention that a high multiplicity of oligonucleotides binds along the target sequence. This is likely to increase the overall efficiency of oligonucleotide probe precursor binding and the subsequent- enzymatic reaction. Moreover, because the oligonucleotide probe precursors are short as discussed above, they are less likely to form intramolecular structures.

Second, ETME and ETML take advantage of highly specific enzymatic processes. In the case of ETME, the high degree of specificity of the polymerase for perfect duplexes essentially serves to “proof-read” the hybridization process by extending (and therefore marking for detection) only those primers that have hybridized to the correct target sequence. This “proof-reading” is likely to increase the overall specificity of the assay over that which can be obtained by unassisted hybridization methods. Both the efficiency and specificity of hybridization is likely to be increased by the ligase enzyme in ETML as well.

Third, unlike surface-base array hybridization systems that rely on the detection of the hybridization event itself, ETME and ETML can mark for detection even transiently stable primer-target interactions. The lifetime of the interaction between the oligonucleotide probe precursors and the target only needs to be long enough to be recognized and acted upon by the polymerase or ligase. This allows a given target sequence to act as a template for multiple precursor binding and subsequent extension or ligation reactions. This process, and the ability to detect transient events, can increase the overall detection sensitivity of the methods over that which can be obtained using unassisted surface-based hybridization assays. As discussed above, this type of reaction can be externally facilitated by artificially cycling the temperature during the extension or ligation reaction.

Finally, the extension or ligation products resulting from methods have a mass range that is greater than that of the oligonucleotide probe precursors. Thus, the spectral peaks resulting from unreacted precursors should not interfere with the mass spectral signature of desired extension or ligated products. In the case of ETME, it is also contemplated that mass-modified ddNTP's may be utilized. This allows for greater assay flexibility and enables multiplexing of the mass analysis step. It is also contemplated that ionization tags may be incorporated into the oligonucleotide probe precursors through a 5′ or 3′-linker, directly into the base or sugar or into the chain-terminating nucleotides. These attributes should help to increase the overall sensitivity of the assays and help to simplify or possibly eliminate separation steps, which will facilitate assay automation and sample throughput.

The present invention also has application to RNAse Protection Assays, which are discussed above and described in Hot-Lines (PharMingen Inc.) Vol. 4, No 1, 1998. In these assays the lengths of the probes are defined so that a particular length corresponds to a particular gene of interest. Probes can also be designed so that the duplexes that register the hybridization events differ on the basis of mass. This will allow for much greater multiplexing capability and make this method applicable for larger sets of genes of interest. The use of gel electrophoresis and blotting are also avoided.

Design of Oligonucleotide Precursors

The design algorithm used in the present invention for the design of oligonucleotide precursors addresses considerations such as sensitivity, specificity, optimization of the multiplexing rate and the like. The design algorithm takes into consideration the various characteristics of the oligonucleotide precursors as discussed above. In the following discussion design of oligonucleotide precursors employed in ETME is emphasized. This is by way of illustration and not limitation. The discussion below applies also to the design of oligonucleotide precursors for ETML and other assays with which the present invention may be used.

A number of issues are considered in the design algorithm including mass coincidence, probe to probe priming, specificity, sensitivity and so forth. Mass coincidence is defined as two different DNA sequences having the same mass. Given a fixed set of mass modifying nucleotides, a number ● representing the length of precursors and a number s representing the number of genes to be interrogated in the assay, the function [symbol] (●,s) is defined. This function measures the probability that s randomly drawn sequences of length ● can be mass modified (using the prescribed mass modifiers) so that all products have different masses. The random set is drawn by uniformly and independently drawing each of its elements. Usually, there will be several oligonucleotide precursor candidates for every gene of interest, which pass the appropriate specificity and sensitivity filters. Such filters are described in Mitsuhashi, et al., and in Shannon, et al (U.S. Pat. No. 6,251,588), the disclosure of which is incorporated herein by reference. The function [symbol] (●,s,k) measures the probability that for s randomly drawn sets of sequences of length ● with k elements in each, there is a set of representatives that can be mass modified (using the prescribed mass modifiers) so that all products have different masses.

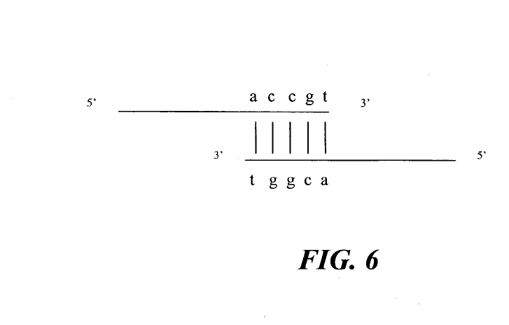

If two oligonucleotide precursors pᵢ, pⱼ have reverse complementary opposite ends as depicted FIG. 6, then, a polymerase may extend each of them with a single nucleotide in a reaction that is not mediated by target T1. Such products will contribute to the mass peaks at mᵢ, mⱼ, skewing the measurement results because not all contribution to these peaks will come from the corresponding-target-mediated extension of the corresponding oligonucleotide precursor. The event described above is designated ETME probe to probe cross priming. This will not occur if the length of the depicted overlap is less than some threshold that can be experimentally determined. Given a number ♦ representing the length of allowable overlap and a number s representing the number of genes to be interrogated in the assay, the function [symbol] (♦,s) may be defined. This function measures the probability that s randomly drawn sequences of length ♦ don't have a reverse complementary pair. The random set is drawn by uniformly and independently drawing each of its elements. Again, several oligonucleotide precursor candidates are available for each gene of interest. The function [symbol] (♦,s,k) measures the probability that, for s randomly drawn sets of sequences of length ♦ with k elements in each, a set of representatives may be found that contains no reverse complementary pair.

For a small enough s, [symbol] (●,s)≧90% and [symbol] (♦,s)≧90%. In this case sets of precursors that satisfy the conditions set forth above for the oligonucleotide precursors may be identified readily, providing that the length ● was large enough so that there is a high probability that none of the oligonucleotide precursors pᵢ has a close match complement in the total mRNA pool of the organism of interest. In this case the design process may focus on the usual sensitivity and specificity issues. In the case when either [symbol] (●,s) or [symbol] (♦,s) is in a medium range (about 50% to about 90%), most candidate sets will not be appropriate, in terms of mass coincidence and probe to probe priming. In the latter situation, a more involved process for finding the appropriate oligonucleotide precursors is necessary, possibly taking priority over the usual sensitivity and specificity issues. For these reasons the behavior of [symbol] (●,s) and z,902 (♦,s) is studied, as there is a close relationship with the size of sets of genes that can be interrogated by the disclosed methods.

An example of a design algorithm is presented below by way of illustration and not limitation.

1. Given the target nucleic acid sequences of interest and the background message, candidate oligonucleotide probe precursors for each gene may be selected based on methods used for selecting hybridization probes. Such methods include, by way of example, PCR primer design software applications (e.g., OLIGO®), neural networks, PCR primer design applications that search for sequences that possess minimal ability to cross-hybridize with other targets present in a sample (e.g., HYBsimulator™), approaches that attempt to predict the efficiency of antisense sequence suppression of m-RNA translation from a combination of predicted nucleic acid duplex melting temperature and predicted target strand structure, homology and predictive secondary structure and Tm information as disclosed in U.S. Pat. No. 6,251,588 (Shannon, et al.), and so forth.

2. Uniformly (or with some weights taking the former sensitivity/specificity ranking into account) a random set of probes is selected, one for each gene of interest.

3. If [symbol] (♦,s) is large (>95%), then this set probably satisfies the probe to probe non-priming condition. Otherwise, a pair of interfering probes is found and changes are made to one of them to avoid the problem. The method is iterated until done. If [symbol] (♦,s) is moderate (>80%), this process should converge to a set satisfying the probe to probe non-priming condition.

4. On the set selected as above, a mass assignment optimization algorithm is run such as that disclosed in U.S. Pat. No. 6,218,118 (Sampson et al.), the relevant disclosure therein being incorporated herein by reference thereto.

5. If [symbol] (●,s) is large (>90%), there is a high probability that a mass assignment with no

mass coincidence is obtained. If not, then a pair of mass coinciding probes is found and the probe selection is changed accordingly without affecting the non-priming condition. The method is reiterated until done, i.e., until a set of probe precursors with no mass coincidence is found.

Analysis Step

Following the above steps, the oligonucleotide products are analyzed by means of mass spectrometry. The details of the analysis are known in the art and will not be repeated here. Suitable mass spectrometers are described in Methods in Enzymology, B. Karger & W. Hancock (editors), Academic Press, San Diego, V270 (1996) and Methods in Enzymology, J. McCloskey (editor), Academic Press, San Diego, V193 (1990). These include matrix assisted laser desorption/ionization (“MALDI”), electrospray (“ESI”), ion cyclotron resonance (“ICR”), Fourier transform types and delayed ion extraction and combinations or variations of the above. Suitable mass analyzers include magnetic sector/magnetic deflection instruments in single quadrupole, triple (“MS/MS”) quadrupole, Fourier transform and time-of-flight (“TOF”) configurations and the like.

It is contemplated that the reaction products may be purified prior to mass spectral analysis using techniques, such as, for example, high performance liquid chromatography (HPLC), capillary electrophoresis and the like. Reverse phase HPLC may be employed to separate the extended or ligated oligonucleotide products according to hydrophobicity. The resulting HPLC fractions may then be analyzed via mass spectrometry. Such techniques may significantly increase the resolving power of the claimed methods.

For analysis by MALDI or the like, it is sometimes desirable to modify the oligonucleotide probe precursors or the oligonucleotide products to impart desirable characteristics to the analysis. Examples of such modifications include those made to decrease the laser energy required to volatilize the oligonucleotides, minimize the fragmentation, create predominantly singly charged ions, normalize the response of the desired oligonucleotides regardless of composition or sequence reduce the peak width, and increase the sensitivity and/or selectivity of the desired analysis product. For example, modifying the phosphodiester backbone of the oligonucleotides via cation exchange may be useful for eliminating peak broadening due to heterogeneity in the cations bound per nucleotide unit. Alternatively, the charged phosphodiester backbone of the oligonucleotides can be neutralized by introducing phosphorothioate internucleotide bridges and alkylating the phosphorothioate with alkyliodide, iodoacetamide, β-iodoethanol, or 2,3-epoxy-1-propanol to form a neutral alkylated phosphorothioate backbone.

It may also be useful to incorporate nucleotide bases which reduce sensitivity to depurination (fragmentation during mass spectrometry), such as N7- or N9-deazapurine nucleotides, RNA building blocks, oligonucleotide triesters, and nucleotide bases having phosphorothioate functions which can be alkylated as described above and the like.

Data Analysis

After a mass spectrum is obtained, an analysis is performed to yield the information defined by the particular application. For example, assume that the mass mᵢ corresponds to the gene gᵢ, for all i. Assume no cross-hybridization, self-priming, etc. Then, the expression level of gᵢ is linearly proportional, or linearly related, to the intensity at mᵢ.

Kits of the Invention

Another aspect of the present invention relates to kits useful for conveniently performing a method in accordance with the invention. To enhance the versatility of the subject invention, the reagents can be provided in packaged combination, in the same or separate containers, so that the ratio of the reagents provides for substantial optimization of the method. The reagents may each be in separate containers or various reagents can be combined in one or more containers depending on the cross-reactivity and stability of the reagents.

In one embodiment a kit comprises a composition comprising a set of oligonucleotide probe precursors wherein each of the oligonucleotide probe precursors in the set binds specifically to a respective target nucleic acid sequence, the oligonucleotide probe precursors are substantially incapable of hybridizing to one another to produce hybrids capable of enzymatic extension, and the oligonucleotide probe precursors can be mass modified by enzymatic extension to yield oligonucleotide products each having a different mass. The kit further comprises an enzyme having DNA polymerase activity and chain-terminating nucleotide triphosphates.

In another embodiment a kit comprises a mixture as described above and a DNA ligase.

In another embodiment a kit comprises a mixture as described above and a condensing agent.

Another embodiment of the present invention is a kit for carrying out a method as described above. The kit comprises a mixture as described above, a DNA ligase and an array comprising a surface and a multiplicity of nucleic acid sequence probes comprising a cleavable linker attached to the surface and a nucleic acid sequence having a 3′-end and a terminal 5′-phosphate wherein the 3′-end of the nucleic acid sequence is attached to the cleavable linker.

In one aspect a kit comprises a condensing agent, an array comprising a surface and a multiplicity of nucleic acid sequence probes comprising a cleavable linker attached to the surface and a nucleic acid sequence having a 3′-end and a terminal 5′-phosphate wherein the 3′-end of the nucleic acid sequence is attached to the cleavable linker.

The kit can further include other separately packaged reagents for conducting the method as well as ancillary reagents and so forth. The relative amounts of the various reagents in the kits can be varied widely to provide for concentrations of the reagents that substantially optimize the reactions that need to occur during the present method.

Under appropriate circumstances one or more of the reagents in the kit can be provided as a dry powder, usually lyophilized, including excipients, which on dissolution will provide for a reagent solution having the appropriate concentrations for performing a method in accordance with the present invention. The kit can further include a written description of a method in accordance with the present invention as described above.

The reagents, methods and kits of the invention are useful for, among others, gene expression profiling and the like. More specifically, the reagents, methods and kits of the invention are useful for, among others, diagnostic and treatment assignment assays based on gene expression profiling. More specifically, the reagents, methods and kits of the invention are useful in, among others, assays designed to determine differential treatment in cancer.

It should be understood that the above description is intended to illustrate and not limit the scope of the invention. Other aspects, advantages and modifications within the scope of the invention will be apparent to those skilled in the art to which the invention pertains. The following examples are put forth so as to provide those of ordinary skill in the art with examples of how to make and use the method and products of the invention, and are not intended to limit the scope of what the inventors regard as their invention.

### EXAMPLES

The invention is demonstrated further by the following illustrative examples. An appropriate computer is employed to carry out the processes discussed below.

### Design of a 2-component Ligation Based Assay

A. This is a general case working with several probe candidates and aiming for a high multiplexing rate.

1. The length of the oligonucleotide probes is determined and represented as λ (divisible by 2). For example, λ=20. The number of genes, s, to be analyzed is determined to satisfy the equation s²L/4^{λ}<0.05, where L is the combined length of the expressed message, i.e., the sum of the lengths of all RNA sequences of the organism to be studied. Next, k is determined such that φ(λ,s,k)≧95%. Values for φ(λ,s,k) are taken from a table compiled from simulations. The candidate oligonucleotide probes are screened for specificity and sensitivity using a method such as those discussed above to find about k precursor candidates, of length λ, in each gene sequence.

2. The following steps are carried out until done, i.e., until a set of probes with no mass coincidence is found or until a MaxNum (for example, 1000) possible sets of representative precursors are tried. A set of representatives, one precursor for each gene, is randomly chosen from the lists of k precursors per gene computed above. A mass modification is attempted that yields mass non-coincidence for this chosen set. By mass non-coincidence is meant that no two probes, pertaining to two different genes, have the same molecular mass. In this step a mass assignment optimization process may be employed such as described in Sampson, et al. Alternatively, standard algorithmic approaches such as, for example, a greedy process, a matching based approach, and the like, may be used.

3. If the above steps fail, i.e. the MaxNum is reached and no appropriate set of probes is found, then the process set forth in paragraph 1 above is repeated and k is increased. Alternatively, the number of genes to be interrogated is reduced. Usually, the reduction is about 10 to about 20%. All possible s² in-order combination of λ/2-long precursors is checked for mass-specificity on the documented coding part of the genome of interest. This is explained more fully as follows. For each gene gᵢ there are two corresponding precursors pᵢ and qᵢ. The oligonucleotide that has the sequence pᵢqᵢ is complementary to a subsequence of the mRNA associated with gᵢ. For all i≠j consider the sequence pᵢqⱼ. If m(pᵢqⱼ)≠m(pᵢqᵢ) for all i, then do nothing. If not, then find pᵢqⱼ's best match in the database containing all known expressed sequences of the target organism. If this introduces a high cross-hybridization potential, then there is a mass-specificity problem.

4. If the aforementioned process fails, i.e., if a match that constitutes a potential cross-hybridization is encountered, then the process of paragraph 2 is repeated.

5. The above processes are repeated until a set of oligonucleotide probes and precursors is selected. The output of the process consists of the set and the corresponding mass assignments.

If λ/2<τ is used in the above experiments, then it is not necessary to screen the set of precursors against precursor to precursor priming. If the steps of paragraph 3 fail several times, e.g., about 3 to about 5 times, then the entire process is repeated using a reduced set of genes to be studied. Usually, the reduction is about 10% to about 20%.

B. This is a specific case working with 100 target genes.

1. The length of the oligonucleotide probes is determined and represented as λ (divisible by 2). Next, check that 10⁴L/4^{λ}<0.05, where L is the combined length of the expressed message of the organism to be studied. For example, λ=16 will work for humans.

2. The candidate oligonucleotide probes are screened for specificity and sensitivity using a method such as those discussed above to find one precursor candidate of length λ in each target gene sequence.

3. A mass modification is attempted that yields mass non-coincidence for this chosen set using a process as discussed above.

4. If the aforementioned process fails, i.e., if mass non-coincidence cannot be attained, then, the process of paragraph 2 is repeated using a different set of probe candidates.

5. All 104 possible in-order combination of λ/2-long precursors are checked for mass-specificity on the documented coding part of the genome of interest. For each gene gᵢ there are two corresponding precursors pᵢ and qᵢ. The oligonucleotide that has the sequence pᵢqᵢ is complementary to a subsequence of the mRNA associated with gᵢ. For all i≠j consider the sequence pᵢqⱼ. If m(pᵢqⱼ)≠m(pᵢqᵢ) for all i, then do nothing. If not, then find pᵢqⱼ's best match in the database containing all known expressed sequences of the target organism. If this introduces a high cross-hybridization potential, then there is a mass-specificity problem.

6. If the process of paragraph 5 fails, i.e., if there is a mass-specificity problem, then, the process of paragraph 3 is repeated using a different set of probe candidates.

7. The above processes are repeated until a set of oligonucleotide probes and precursors is selected. The output of the process comprises 200 precursor sequences of length 8 in this example (2 for each target gene), the mass mᵢ that corresponds to each gene gᵢ (a hundred different masses), and the mass modifications needed to obtain specificity.

What is claimed is:

1. A method of quantitatively analyzing a set of target nucleic acid sequences, said method comprising:
(a) hybridizing a set of oligonucleotide probe precursors to said target nucleic acid sequences to produce hybrids, wherein a unique set of oligonucleotide precursors is employed for each set of target nucleic acid sequences,
(b) processing said hybrids to alter the mass of each of said oligonucleotide probe precursors in said hybrids in a target sequence-mediated reaction to produce oligonucleotide products, each of which has a unique mass characteristic of its respective target nucleic acid sequence, which unique mass is not a result of the presence of a mass tag in said oligonucleotide product, and
(c) analyzing said oligonucleotide products by means of mass spectrometry and relating the results thereof to the amount of said target nucleic acid sequences in said set.

2. A method according to claim 1 wherein the composition of said set of target nucleic acid sequences is known.

3. A method according to claim 1 further comprising purifying said oligonucleotide products prior to said analyzing.

4. A method according to claim 1 further comprising separating said oligonucleotide products prior to said analyzing.

5. A method according to claim 1 wherein steps (a) and (b) are conducted in solution.

6. A method according to claim 1 wherein steps (a) and (b) are conducted with a surface-bound set of oligonucleotide probes.

7. A method according to claim 1 wherein said oligonucleotide products are analyzed by means of MALDI-TOF mass spectrometry.

8. A method according to claim 1 wherein said processing comprises a target sequence mediated enzymatic approach.

9. A method according to claim 8 wherein said enzymatic approach is selected from the group consisting of polymerase extension and ligation.

10. A method according to claim 1 wherein said processing comprises extending each of said hybridized oligonucleotide probe precursors by polymerizing at least one nucleotide at the 3′-end of said hybridized oligonucleotide probe precursors.

11. A method according to claim 10 wherein said polymerizing utilizes an enzyme having DNA polymerase activity.

12. A method according to claim 10 wherein said nucleotide is a chain-terminating nucleotide triphosphate.

13. A method according to claim 1 wherein said processing comprises ligating adjacent oligonucleotide probe precursors.

14. A method according to claim 13 wherein said ligating involves a DNA ligase.

15. A method according to claim 13 wherein said processing comprises ligating adjacent oligonucleotide probe precursors using a condensing agent.

16. A method according to claim 1 wherein said set of target nucleic acid sequences is a set of mRNA's.

17. A method according to claim 1 wherein said oligonucleotide probe precursors are selected by a process comprising:
(a) screening oligonucleotides to identify oligonucleotide probe precursors that bind specifically to each of said target nucleic acid sequences,
(b) screening said oligonucleotide probe precursors to select oligonucleotide probe precursors that are substantially incapable of hybridizing to one another to form hybrids that are enzymatically extendable, and
(c) screening said oligonucleotide probe precursors to select oligonucleotide probe precursors that can be mass modified by enzymatic extension to yield oligonucleotide products each having a different mass.

18. A method of determining the expression of genes in a set of genes, said method comprising:
(a) hybridizing a set of oligonucleotide probe precursors to said set of genes to produce hybrids, wherein (i) the composition of each of said genes is known to the extent necessary to select said set of oligonucleotide probe precursors and (ii) each of said oligonucleotide probe precursors has a unique mass, binds specifically to a respective gene, is substantially incapable of hybridizing to another of said oligonucleotide probe precursors to produce a hybrid capable of enzymatic extension, and is modifiable by enzymatic extension to yield oligonucleotide products each having a unique mass that is not a result of the presence of a mass tag in said oligonucleotide product,
(b) extending each of said hybridized oligonucleotide probe precursors by polymerizing at least one nucleotide at the 3′-end of said hybridized oligonucleotide probe precursors to produce said oligonucleotide products, and
(c) analyzing said oligonucleotide products by means of mass spectrometry and relating the results thereof to said expression of said genes in said set.

19. A method according to claim 18 wherein each of said oligonucleotide probe precursors has a length of about 15 to about 30 nucleotides.

20. A method according to claim 18 wherein said polymerizing utilizes an enzyme having DNA polymerase activity.

21. A method according to claim 18 wherein said nucleotide is a chain-terminating nucleotide triphosphate.

22. A method according to claim 18 wherein said oligonucleotide probe precursors are selected by a process comprising:
(a) screening oligonucleotides to identify oligonucleotide probe precursors that bind specifically to each of said genes,
(b) screening said oligonucleotide probe precursors to select oligonucleotide probe precursors that are substantially incapable of hybridizing to one another to form hybrids that are enzymatically extendable, and
(c) screening said oligonucleotide probe precursors to select oligonucleotide probe precursors that can be mass modified by enzymatic extension to yield oligonucleotide products each having a different mass.

23. A method of determining the expression of genes in a set of genes, said method comprising:
(a) hybridizing a set of oligonucleotide probe precursors to said genes to produce hybrids, wherein (i) the composition of each of said genes is known to the extent necessary to select said set of oligonucleotide probe precursors and (ii) at least two of said oligonucleotide probe precursors in said set bind specifically and adjacently to a respective gene and are ligatable to yield oligonucleotide products each having a different mass that is'not a result of the presence of a mass tag in said oligonucleotide product,
(b) ligating adjacent oligonucleotide probe precursors to produce oligonucleotide products, each of which has a unique mass and
(c) analyzing said oligonucleotide products by means of mass spectrometry and relating the results thereof to the amount of expression of said genes in said set.

24. A method according to claim 23 wherein each of said oligonucleotide probe precursors is about 6 to about 8 nucleotides in length,

25. A method according to claim 23 wherein said ligating involves a DNA ligase.

26. A method according to claim 23 wherein said processing comprises ligating adjacent oligonucleotide probe precursors using a condensing agent.

27. A method according to claim 23 wherein said set of oligonucleotide probe precursors is selected so that two of said oligonucleotide probe precursors in said set bind specifically and adjacently to a respective gene.

28. A method according to claim 23 wherein said set of oligonucleotide probe precursors is selected so that three of said oligonucleotide probe precursors in said set bind specifically and adjacently to a respective gene.

29. A composition comprising a set of oligonucleotide probe precursors characterized as follows:
(a) each of said oligonucleotide probe precursors in said set binds specifically to a respective target nucleic acid sequence,
(b) said oligonucleotide probe precursors are substantially incapable of hybridizing to one another to produce hybrids capable of enzymatic extension, and
(c) said oligonucleotide probe precursors can be mass modified by enzymatic extension to yield oligonucleotide products each having a different mass that is not a result of the presence of a mass tag in said oligonucleotide product.

30. A composition according to claim 29 wherein the length of said oligonucleotide probe precursors is about 15 to about 30 nucleotides.

31. A kit for analyzing a set of target nucleic acid sequences, said kit comprising in packaged combination:
(a) a composition according to claim 30,
(b) an enzyme having DNA polymerase activity; and
(c) chain-terminating nucleotide triphosphates.

32. A kit according to claim 31 further comprising a set of oligonucleotide probes attached to a surface of a support.

33. A kit according to claim 32 wherein the 3′-end of said probes is attached to said surface by cleavable linkers.

34. A composition comprising a set of oligonucleotide probe precursors characterized as follows:
(a) each of said oligonucleotide probe precursors has a length of about 5 to about 10 nucleotides and
(b) at least three of said oligonucleotide probe precursors in said set bind specifically to a respective target nucleic acid sequence and are ligatable to yield an oligonucleotide product having a different mass that is not a result of the presence of a mass tag in said oligonucleotide product.

35. A kit according to claim 34 wherein each of said oligonucleotide probe precursors has a length of about 6 to about 7 nucleotides.

36. A kit for analyzing a set of target nucleic acid sequences, said kit comprising in packaged combination:
(a) a composition according to claim 34 and
(b) a DNA ligase or a condensing agent for ligating said oligonucleotide probe precursors.

37. A kit according to claim 36 further comprising a set of oligonucleotide probes attached to a surface of a support.

38. A kit according to claim 37 wherein said probes are attached to said surface by cleavable linkers.

39. A kit according to claim 36 wherein one of said three oligonucleotide probe precursors is attached to a surface of a support.

40. A method of determining the expression of genes in a set of genes, said method comprising:
(1) hybridizing said set of genes to a multiplicity of nucleic acid probes attached to a surface in an array wherein said multiplicity of nucleic acid sequence probes comprise (i) a cleavable linker attached to said surface and (ii) a nucleic acid sequence having a 3′-end and a terminal 5′-phosphate wherein said 3′-end of said nucleic acid sequence is attached to said cleavable linker;
(2) hybridizing a set of oligonucleotide probe precursors to said genes, wherein a unique set of oligonucleotide precursors is employed for each set of genes,
(3) processing said hybrids to alter the mass of each of said oligonucleotide probe precursors in said hybrids in a target sequence-mediated reaction to produce oligonucleotide products, each of which has a unique mass that is not a result of the presence of a mass tag in said oligonucleotide product,
(4) cleaving said cleavable linker; and
(5) analyzing said oligonucleotide products by mass spectrometry and relating the results thereof to the amount of said genes in said set.

41. A method according to claim 40 wherein the composition of said set of genes is known.

42. A method according to claim 40 wherein said oligonucleotide products are analyzed by means of MALDI-TOF mass spectrometry.

43. A method according to claim 40 wherein said processing comprises a target sequence mediated enzymatic approach.

44. A method according to claim 43 wherein said enzymatic approach is selected from the group consisting of polymerase extension and ligation.

45. A method according to claim 40 wherein said oligonucleotide probe precursors are selected by a process comprising:
(a) screening oligonucleotides to identify oligonucleotide probe precursors that bind specifically to each of said genes,
(b) screening said oligonucleotide probe precursors to select oligonucleotide probe precursors that are substantially incapable of hybridizing to one another to form hybrids that are enzymatically extendable, and
(c) screening said oligonucleotide probe precursors to select oligonucleotide probe precursors that can be mass modified by enzymatic extension to yield oligonucleotide products each having a different mass.
